(19) United States
(12) Patent Application Publication
(10) Pub. No.: US 2025/0325492 A1
(43) Pub. Date: Oct. 23, 2025

(54) IONIZABLE LIPIDS, LIPID NANOPARTICLES FOR MRNA DELIVERY AND METHODS OF MAKING THE SAME

(71) Applicant: University of Cincinnati, Cincinnati, OH (US)

(72) Inventors: Joo-Youp Lee, Cincinnati, OH (US); Vishnu Sriram, Cincinnati, OH (US)

(73) Assignee: University of Cincinnati, Cincinnati, OH (US)

(21) Appl. No.: 18/870,871

(22) PCT Filed: Jun. 2, 2023
(86) PCT No.: PCT/US2023/024325

§ 371 Date: Dec. 2, 2024

Related U.S. Application Data
(60) Provisional application No. 63/348,737, filed on Jun. 3, 2022.
(60) Provisional application No. 63/354,479, filed on Jun. 22, 2022.
(60) Provisional application No. 63/390,827, filed on Jul. 20, 2022.

(51) Int. Cl. A61K 9/51; A61K 31/7105

(52) U.S. Cl. CPC A61K 9/5123; A61K 31/7105

(57) ABSTRACT

A composition including ionizable lipids is provided. Methods of making the ionizable lipids are also provided. Also provided are compositions forming lipid nanoparticles, wherein the composition includes the ionizable lipid, a helper lipid, a structural lipid or sterol, and a polymer-conjugated lipid. Methods of using the ionizable lipid and lipid nanoparticles are also provided.

### CROSS REFERENCE TO RELATED APPLICATIONS

This application claims priority to U.S. Provisional Application Ser. No. 63/348,737, filed Jun. 3, 2022, U.S. Provisional Application Ser. No. 63/354,479, filed Jun. 22, 2022, and U.S. Provisional Application Ser. No. 63/390,827, filed Jul. 20, 2022, the entire contents of which are incorporated herein by reference.

### TECHNICAL FIELD

The present disclosure generally relates to ionizable lipids, lipid nanoparticles, and methods of making and using the same.

### BACKGROUND

One of the major challenges in the field of targeted delivery of biologically active substances is their instability and low cell penetrating potential, as well as their susceptibility to enzymatic degradation. This has created challenges in the development of therapies utilizing nucleic acid molecules, in particular RNA molecules.

In that respect, lipid-based nanoparticle compositions such as lipoplexes and liposomes have been used as packaging vehicles for biologically active substances to allow transport into cells and/or intracellular compartments. These lipid-based nanoparticle compositions typically comprise a mixture of different lipids such as ionizable lipids, helper lipids, structural lipids (such as sterols or cholesterol), and lipid conjugates.

Emerging clinical therapies, particularly nucleic acid-based vaccines, require drug delivery systems, such lipid nanoparticles, that can encapsulate and deliver a variety of cargo molecules. Accordingly, a need exists to develop new lipids and/or nanoparticles to better deliver the therapy.

### SUMMARY

According to embodiments, a composition includes at least one ionizable lipid according to General Formula (I),

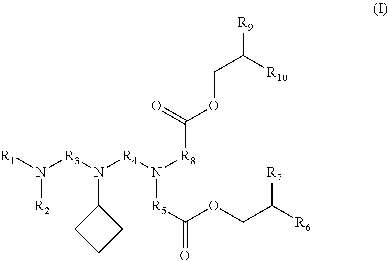

or a pharmaceutically-acceptable salt thereof, in which: R¹ is independently selected from the group consisting of C1 to C2 alkyl groups; R2 is independently selected from the group consisting of C1 to C2 alkyl groups; R3 is independently selected from the group consisting of C2 to C4 alkyl groups; R4 is independently selected from the group consisting of C2 to C4 alkyl groups; R5 is independently selected from the group consisting of C2 to C8 alkyl groups; R6 is independently selected from the group consisting of C1 to C12 alkyl groups; R7 is independently selected from the group consisting of C1 to C12 alkyl groups; R8 is independently selected from the group consisting of C2 to C8 alkyl groups; R9 is independently selected from the group consisting of C1 to C12 alkyl groups; and R10 is independently selected from the group consisting of C1 to C12 alkyl groups.

According to other embodiments, a composition includes at least one ionizable lipid according to General Formula (II),

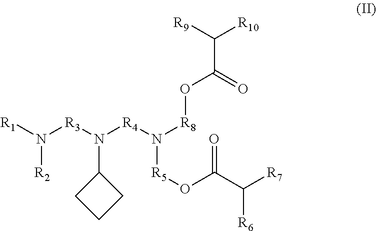

or a pharmaceutically-acceptable salt thereof, in which: R₁ is independently selected from the group consisting of C₁ to C₂ alkyl groups; R₂ is independently selected from the group consisting of C₁ to C₂ alkyl groups; R₃ is independently selected from the group consisting of C₂ to C₄ alkyl groups; R₄ is independently selected from the group consisting of C₂ to C₄ alkyl groups; R₅ is independently selected from the group consisting of C₂ to C₈ alkyl groups; R₆ is independently selected from the group consisting of —H, and C₁ to C₁₂ alkyl groups; R₇ is independently selected from the group consisting of C₁ to C₁₂ alkyl groups; R₈ is independently selected from the group consisting of C₂ to C₈ alkyl groups; R₉ is independently selected from the group consisting of —H and C₁ to C₁₂ alkyl groups; and R₁₀ is independently selected from the group consisting of C₁ to C₁₂ alkyl groups.

According to other embodiments, a composition includes at least one ionizable lipid according to General Formula (III),

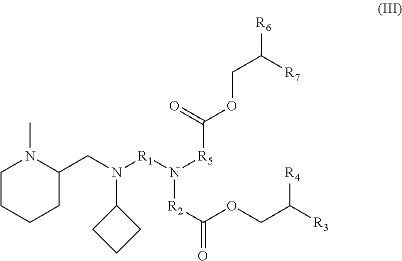

or a pharmaceutically-acceptable salt thereof, in which: R₁ is independently selected from the group consisting of C₂ to C₄ alkyl groups; R₂ is independently selected from the group consisting of C₂ to C₈ alkyl groups; R₃ is independently selected from the group consisting of C₁ to C₁₂ alkyl groups; R₄ is independently selected from the group consisting of C₁ to C₁₂ alkyl groups; R₅ is independently selected from the group consisting of C₂ to C₈ alkyl groups; R₆ is independently selected from the group consisting of C₁ to C₁₂ alkyl groups; and R₇ is independently selected from the group consisting of C₁ to C₁₂ alkyl groups.

According to other embodiments, a composition includes at least one ionizable lipid according to General Formula (IV),

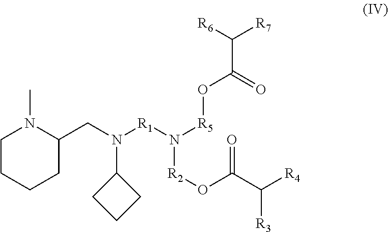

or a pharmaceutically-acceptable salt thereof, in which: R₁ is independently selected from the group consisting of C₂ to C₄ alkyl groups; R₂ is independently selected from the group consisting of C₂ to C₈ alkyl groups; R₃ is independently selected from the group consisting of C₁ to C₁₂ alkyl groups; R₄ is independently selected from the group consisting of —H and C₁ to C₁₂ alkyl groups; R₅ is independently selected from the group consisting of C₂ to C₈ alkyl groups; R₆ is independently selected from the group consisting of —H, and C₁ to C₁₂ alkyl groups; and R₇ is independently selected from the group consisting of C₁ to C₁₂ alkyl groups.

According to other embodiments, a composition includes at least one ionizable lipid, a helper lipid; a structural lipid or sterol; and a polymer-conjugated lipid, wherein the composition forms lipid nanoparticles.

These and other features, aspects, and advantages of the present invention will become better understood with reference to the following description, appended claims, and accompanying drawings.

### BRIEF DESCRIPTION OF THE DRAWINGS

Though the specification concludes with claims particularly pointing out and distinctly claiming the invention, it is believed that the present invention will be better understood from the following description taken in conjunction with the accompanying drawings, in which:

FIG. 1 depicts a schematic of the synthesis of 1,1′-bis(2-hexyldecyl) 6,6′-[(2-chloroethyl)imino]bis[hexanoate], according to one or more embodiments shown and described herein;

FIG. 2 depicts an ¹H NMR spectrum of a first intermediate product (Product A) in the synthesis depicted in FIG. 1, according to one or more embodiments shown and described herein;

FIG. 3 depicts an ¹H NMR spectrum of a second intermediate product (Control 1) in the synthesis depicted in FIG. 1, according to one or more embodiments shown and described herein;

FIG. 4 depicts an ¹H NMR spectrum of 1,1′-bis(2-hexyldecyl) 6,6′-[(2-chloroethyl)imino]bis[hexanoate](Product B) in the synthesis depicted in FIG. 1, according to one or more embodiments shown and described herein;

FIG. 5 depicts a schematic of the synthesis of 1,1′-bis(2-hexyldecyl) 6,6′-[[2-(dimethylamino)ethyl]imino]bis[hexanoate](Control 2), according to one or more embodiments shown and described herein;

FIG. 6 depicts an ¹H NMR spectrum of Control 2 in the synthesis depicted in FIG. 5, according to one or more embodiments shown and described herein;

FIG. 7 depicts an illustrative embodiment of the synthesis of Lipid 1, according to one or more aspects shown and described herein;

FIG. 8 depicts an ¹H NMR spectrum of Intermediate 1 in the synthesis depicted in FIG. 7, according to one or more embodiments shown and described herein;

FIG. 9 depicts an ¹H NMR spectrum of Lipid 1 in the synthesis depicted in FIG. 7, according to one or more embodiments shown and described herein;

FIG. 10 depicts an illustrative embodiment of the synthesis of Lipid 2, according to one or more embodiments shown and described herein;

FIG. 11 depicts an ¹H NMR spectrum of Intermediate 2, in the synthesis depicted in FIG. 10, according to one or more embodiments shown and described herein;

FIG. 12 depicts an ¹H NMR spectrum of Lipid 2, in the synthesis depicted in FIG. 10, according to one or more embodiments shown and described herein;

FIG. 13 depicts an illustrative embodiment of the synthesis of Lipid 3, according to one or more embodiments shown and described herein;

FIG. 14 an ¹H NMR spectrum of Intermediate 3, in the synthesis depicted in FIG. 13, according to one or more embodiments shown and described herein;

FIG. 15 depicts an ¹H NMR spectrum of Lipid 3, in the synthesis depicted in FIG. 13, according to one or more embodiments shown and described herein;

FIG. 16A depicts a graphical representation of the particle size of various lipid nanoparticles according to one or more embodiments shown and described herein;

FIG. 16B depicts a graphical representation of the surface charge of various lipid nanoparticles according to one or more embodiments shown and described herein;

FIG. 17A depicts a graphical representation of cell uptake of various lipid nanoparticle formulations in Jurkat cells, according to one or more embodiments shown and described herein;

FIG. 17B depicts a graphical representation of transfection efficiency of various lipid nanoparticle formulations in Jurkat cells, according to one or more embodiments shown and described herein.

The exemplifications set out herein illustrate at least one embodiment of the present disclosure, and such exemplifications are not to be construed as limiting the scope of the present disclosure in any manner.

### DETAILED DESCRIPTION

Features and advantages of the invention will now be described with occasional reference to specific embodiments. However, the invention may be embodied in different forms and should not be construed as limited to the embodiments set forth herein. Rather, these embodiments are provided so that this disclosure will be thorough and complete and will fully convey the scope of the invention to those skilled in the art.

Unless otherwise defined, all technical and scientific terms used herein have the same meaning as commonly understood by one of ordinary skill in the art to which the invention belongs. The terminology used in the description herein is for describing particular embodiments only and is not intended to be limiting.

As used herein, the singular forms “a,” “an,” and “the” are intended to include the plural forms, including “at least one,” unless the content clearly indicates otherwise. “Or” means “and/or.” As used herein, the term “and/or” includes any and all combinations of one or more of the associated listed items. It will be further understood that the terms “comprises” and/or “comprising,” or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof. It is to be further understood that where descriptions of various embodiments use the term “comprising,” and/or “including” those skilled in the art would understand that in some specific instances, an embodiment can be alternatively described using language “consisting essentially of” or “consisting of.” The term “or a combination thereof” means a combination including at least one of the foregoing elements.

Unless otherwise indicated, all numbers expressing quantities of ingredients, properties such as molecular weight, reaction conditions, and so forth as used in the specification and claims are to be understood as being modified in all instances by the term “about.” Accordingly, unless otherwise indicated, the numerical properties set forth in the specification and claims are approximations that may vary depending on the desired properties sought to be obtained in embodiments of the present invention. Notwithstanding that the numerical ranges and parameters setting forth the broad scope of the invention are approximations, the numerical values set forth in the specific examples are reported as precisely as possible. One of ordinary skill in the art will understand that any numerical values inherently contain certain errors attributable to the measurement techniques used to ascertain the values.

It should be understood that every numerical range given throughout this specification will include every narrower numerical range that falls within such broader numerical range, as if such narrower numerical ranges were all expressly written herein. Ranges can be expressed herein as from “about” one particular value, and/or to “about” another particular value. When such a range is expressed, examples include from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

Ranges provided herein are understood to be shorthand for all of the values within the range. For example, a range of 1 to 25 is understood to include any number, combination of numbers, or sub-range from the group consisting of 1, 2, 3, 4, 5, 6, 7, 8, 9, 10, 11, 12, 13, 14, 15, 16, 17, 18, 19, 20, 21, 22, 23, 24, or 25, as well as all intervening decimal values between the aforementioned integers such as, for example, 1.1, 1.2, 1.3, 1.4, 1.5, 1.6, 1.7, 1.8, and 1.9. With respect to sub-ranges, “nested sub-ranges” that extend from either end point of the range are specifically contemplated. For example, a nested sub-range of an exemplary range of 1 to 25 may comprise 1 to 5, 1 to 10, 1 to 15, and 1 to 20 in one direction, or 25 to 20, 25 to 15, 25 to 10, and 25 to 5 in the other direction.

As used herein, the terms “improve,” “increase,” “inhibit,” “reduce,” or grammatical equivalents thereof, indicate values that are relative to a baseline or other reference measurement. In some embodiments, an appropriate reference measurement may be or comprise a measurement in a particular system (e.g., in a single subject) under otherwise comparable conditions absent presence of (e.g., prior to and/or after) a particular agent or treatment, or in presence of an appropriate comparable reference agent. In some embodiments, an appropriate reference measurement may be or comprise a measurement in comparable system known or expected to respond in a particular way, in presence of the relevant agent or treatment.

As used herein, the term “expression” of a nucleic acid sequence refers to the generation of any gene product from the nucleic acid sequence. In some embodiments, a gene product can be a transcript. In some embodiments, a gene product can be a polypeptide. In some embodiments, expression of a nucleic acid sequence involves one or more of the following: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

As used herein, the term “aliphatic” includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, and/or alkynyl moieties. Thus, as used herein, the term “alkyl” includes straight and branched alkyl groups. An analogous convention applies to other generic terms such as “alkenyl,” “alkynyl” and the like. Furthermore, as used herein, the terms “alkyl,” “alkenyl,” “alkynyl” and the like encompass both substituted and unsubstituted groups.

In some embodiments, the alkyl, alkenyl, and alkynyl groups described herein contain from 1 to 15 aliphatic carbon atoms. In other embodiments, the alkyl, alkenyl, and alkynyl groups described herein contain from 1 to 12 aliphatic carbon atoms. In still other embodiments, the alkyl, alkenyl, and alkynyl groups described herein contain from 1 to 8 aliphatic carbon atoms. In yet other embodiments, the alkyl, alkenyl, and alkynyl groups described herein contain from 1 to 4 aliphatic carbon atoms. In some embodiments, the alkyl, alkenyl, and alkynyl groups described herein contain from 1 to 2 aliphatic carbon atoms. In other embodiments, the alkyl, alkenyl, and alkynyl groups described herein contain from 2 to 12 aliphatic carbon atoms. In other embodiments, the alkyl, alkenyl, and alkynyl groups described herein contain from 2 to 8 aliphatic carbon atoms. In still other embodiments, the alkyl, alkenyl, and alkynyl groups described herein contain from 2 to 4 aliphatic carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, neopentyl, sec-pentyl, isopentyl, tert-pentyl, n-hexyl, sec-hexyl, heptyl, octyl, nonyl, decyl, undecyl, dodecyl, moieties and the like, which optionally may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl, and the like.

Some examples of substituents of the above-described aliphatic moieties of compounds described herein include, but are not limited to aliphatic; alicyclic; heteroaliphatic; heterocyclic; amino; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)R^{X}; —CO₂(R^{X}); —CON(R^{X})₂; —OC(O)R^{X}; —OCO₂R^{X}; —OCON(R^{X})₂; —N(R^{X})₂; —S(O)₂R^{X}; —NR^{X}(CO)R^{X}, wherein each occurrence of R^{X} independently includes, but is not limited to, aliphatic, alicyclic, heteroaliphatic, heterocyclic moieties, and wherein any of the aliphatic, alicyclic, heterocyclic, and amino substituents described above herein and may be substituted or unsubstituted, branched or unbranched, and saturated or unsaturated.

In some embodiments, the aliphatic group is substituted by one or more amino groups. In some embodiments, the aliphatic group is substituted by at least two amino groups In some embodiments, the aliphatic group is an alkyl chain substituted by one or more amino groups. In some embodiments, the aliphatic group is an alkyl chain substituted by at least two amino groups. As used herein, the term “amino” refers to a primary amine (—NH₂), a secondary amine (—NHR^{X}), a tertiary amine (—NR^{X}R^{Y}), or a quaternary amine (—N+R^{X}R^{Y}R^{Z}), where R^{X}, R^{Y}, and R^{Z} are independently an aliphatic, alicyclic, heteroaliphatic, heterocyclic, aromatic or heteroaromatic moiety, as defined herein. Examples of amino groups include, but are not limited to, methylamino, dimethylamino, ethylamino, diethylamino, diethylaminocarbonyl, methylethylamino, isopropylamino, piperidino, trimethylamino, and propylamino.

Additional examples of generally applicable substituents are illustrated by the specific embodiments shown in the Examples that are described below.

Unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is no way intended that any particular order be inferred. Any recited single or multiple feature or aspect in any one claim can be combined or permuted with any other recited feature or aspect in any other claim or claims.

The term “independently selected from,” as used herein, is intended to mean that the referenced groups can be the same, different, or a mixture thereof, unless the context clearly indicates otherwise. Thus, under this definition, the phrase “X¹, X², and X³ are independently selected from noble gases” would include the scenario where X¹, X², and X³ are all the same, where X¹, X², and X³ are all different, and where X¹ and X² are the same but X³ is different.

The term “subject” as used herein refers to any living organism to which a pharmaceutical can be administered. The term subject includes, but is not limited to, humans, nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult, child, and newborn subjects, as well as fetuses, whether male or female, are intended to be covered.

As used herein, the term “pharmaceutically acceptable” refers to approved or approvable by a regulatory agency of the Federal or a state government or listed in the U.S. Pharmacopeia or other generally recognized pharmacopeia for use in animals, including humans.

As used herein, the term “pharmaceutically acceptable excipient, carrier, or diluent” or the like refer to an excipient, carrier, or diluent that can be administered to a subject, together with an agent, and which does not destroy the pharmacological activity thereof and is nontoxic when administered in doses sufficient to deliver a therapeutic amount of the agent.

The term “pharmaceutically acceptable salt” as used herein refers to pharmaceutically acceptable organic or inorganic salts of an ionizable lipid of the present disclosure. Exemplary salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucuronate, saccharate, formate, benzoate, glutamate, methanesulfonate “mesylate,” ethanesulfonate, benzenesulfonate, p-toluenesulfonate, pamoate (i.e., 1,1′-methylene-bis-(2-hydroxy-3-naphthoate)) salts, alkali metal (e.g, sodium and potassium) salts, alkaline earth metal (e.g., magnesium) salts, and ammonium salts. A pharmaceutically acceptable salt may involve the inclusion of another molecule such as an acetate ion, a succinate ion or other counter ion. The counter ion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counter ions.

The term “administration” of the pharmaceutically active compounds and the pharmaceutical compositions defined herein includes systemic use, as by parenteral administration, (e.g., injection, intravenous infusion, etc.), suppositories, transdermal administration, nasal, bronchial, or respiratory administration, and oral administration thereof, as well as topical application of the compounds and compositions.

As used herein, the term “lipid encapsulated” is meant to refer to a lipid particle that provides an active agent or therapeutic agent, such as a nucleic acid (e.g, an anti-sense oligonucleotide (ASO), mRNA, siRNA, close ended DNA (ceDNA), viral vector, etc.), with full encapsulation, partial encapsulation, or both. In a preferred embodiment, the nucleic acid is fully encapsulated in the lipid particle (e.g., to form a nucleic acid containing lipid particle).

Unless otherwise stated, the structures depicted and described herein include all isomeric (e.g., enantiomeric, diastereomeric, and geometric) forms of the structure; for example, tautomers, R and S configurations for each asymmetric center, Z and E double bond isomers, and Z and E conformational isomers. Additionally, unless otherwise stated, the structures depicted and described herein include compounds that differ only in the presence of one or more isotopically enriched atoms. For example, compounds having the present structures including the replacement of hydrogen by deuterium or tritium, or the replacement of a carbon by a ¹³C- or ¹⁴C-enriched carbon are within the scope of this invention. Such compounds are useful, for example, as analytical tools or as therapeutic agents.

### Ionizable Lipids

As used herein, the term “ionizable lipid” refers to a lipid, e.g., cationic lipid, having at least one protonatable or deprotonatable group, such that the lipid is positively charged at a pH at or below physiological pH (e.g., pH 7.4), and neutral at a second pH, wherein the second pH is at or above physiological pH. It will be understood by one of ordinary skill in the art that the addition or removal of protons as a function of pH is an equilibrium process, and that the reference to a charged or a neutral lipid refers to the nature of the predominant species and does not require that all of the lipid be present in the charged or neutral form. In some embodiments, an ionizable lipid is characterized by three portions: an amine head, a linker and a hydrophobic tail.

Embodiments herein are directed to ionizable lipids, lipid nanoparticles, and pharmaceutical compositions. The compositions and pharmaceutical compositions contain one or more compounds having General Formula (I), General Formula (II), General Formula (III), or General Formula (IV), or pharmaceutically-acceptable salts thereof.

General Formula (I) has the structure:

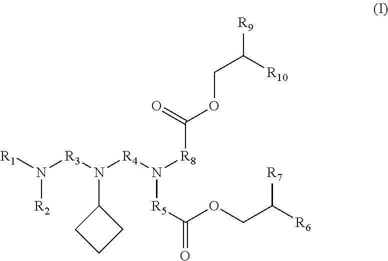

in which any of R₁-R₁₀ is chosen from C₁ to C₂₀ aliphatic groups, wherein the aliphatic group is selected from alkyl, alkenyl, and alkynyl groups. In some embodiments, R₁ is chosen from C₁ to C₂ aliphatic groups, wherein the aliphatic group is selected from alkyl, alkenyl, and alkynyl groups; R₂ is chosen from C₁ to C₂ aliphatic groups, wherein the aliphatic group is selected from alkyl, alkenyl, and alkynyl groups; R₃ is chosen from C₂ to C₄ aliphatic groups, wherein the aliphatic group is selected from alkyl, alkenyl, and alkynyl groups; R₄ is chosen from C₂ to C₄ aliphatic groups, wherein the aliphatic group is selected from alkyl, alkenyl, and alkynyl groups; R₅ is chosen from C₂ to C₈ aliphatic groups, wherein the aliphatic group is selected from alkyl, alkenyl, and alkynyl groups; R₆ is chosen from C₁ to C₂₀ aliphatic groups, wherein the aliphatic group is selected from alkyl, alkenyl, and alkynyl groups; R₇ is chosen from C₁ to C₂₀ aliphatic groups, aliphatic groups, wherein the aliphatic group is selected from alkyl, alkenyl, and alkynyl groups; R₈ is chosen from C₂ to C₂₀ aliphatic groups, wherein the aliphatic group is selected from alkyl, alkenyl, and alkynyl groups; R₉ is chosen from C₁ to C₂₀ aliphatic groups, wherein the aliphatic group is selected from alkyl, alkenyl, and alkynyl groups; R₁₀ is chosen from C₁ to C₂₀ aliphatic groups, aliphatic groups, wherein the aliphatic group is selected from alkyl, alkenyl, and alkynyl groups. In some embodiments, each of R₁-R₁₀ has an alkyl aliphatic group. In some embodiments, the aliphatic group of each of R₁-R₁₀ is independently selected.

In some embodiments, ionizable lipids of the present disclosure have the structure of General Formula (I), wherein R₁ is chosen from C₁ to C₂ alkyl groups; R₂ is chosen from C₁ to C₂ alkyl groups; R₃ is chosen from C₂ to C₄ alkyl groups; R₄ is chosen from C₂ to C₄ alkyl groups; R₅ is chosen from C₂ to C₈ alkyl groups; R₆ is chosen from C₁ to C₁₂ alkyl groups; R₇ is chosen from C₁ to C₁₂ alkyl groups; R₈ is chosen from C₂ to C₈ alkyl groups; R₉ is chosen from C₁ to C₁₂ alkyl groups; and R₁₀ is chosen from C₁ to C₁₂ alkyl groups.

General Formula (II) has the structure:

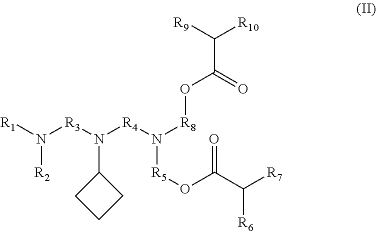

in which R₁ is chosen from C₁ to C₂ aliphatic groups, wherein the aliphatic group is selected from alkyl, alkenyl, and alkynyl groups; R₂ is chosen from C₁ to C₂ aliphatic groups, wherein the aliphatic group is selected from alkyl, alkenyl, and alkynyl groups; R₃ is chosen from C₂ to C₄ aliphatic groups, aliphatic groups, wherein the aliphatic group is selected from alkyl, alkenyl, and alkynyl groups; R₄ is chosen from C₂ to C₄ aliphatic groups, wherein the aliphatic group is selected from alkyl, alkenyl, and alkynyl groups; R₅ is chosen from C₂ to C₈ aliphatic groups, wherein the aliphatic group is selected from alkyl, alkenyl, and alkynyl groups; R₆ is chosen from —H and C₁ to C₂₀ aliphatic groups, wherein the aliphatic group is selected from alkyl, alkenyl, and alkynyl groups; R₇ is chosen from C₁ to C₂₀ aliphatic groups, aliphatic groups, wherein the aliphatic group is selected from alkyl, alkenyl, and alkynyl groups; R₈ is chosen from C₂ to C₂₀ aliphatic groups, wherein the aliphatic group is selected from alkyl, alkenyl, and alkynyl groups; R₉ is chosen from —H and C₁ to C₂₀ aliphatic groups, wherein the aliphatic group is selected from alkyl, alkenyl, and alkynyl groups; R₁₀ is chosen from C₁ to C₂₀ aliphatic groups, aliphatic groups, wherein the aliphatic group is selected from alkyl, alkenyl, and alkynyl groups. In some embodiments, each of R₁-R₁₀ has an alkyl aliphatic group. In some embodiments, each of R₁-R₅, R₇—R₈ and R₁₀ have an alkyl aliphatic group. In some embodiments, the aliphatic group of each of R₁-R₁₀ is independently selected.

In some embodiments, ionizable lipids of the present disclosure have the structure of General Formula (II), wherein R₁ is chosen from C₁ to C₂ alkyl groups; R₂ is chosen from C₁ to C₂ alkyl groups; R₃ is chosen from C₂ to C₄ alkyl groups; R₄ is chosen from C₂ to C₄ alkyl groups; R₅ is chosen from C₂ to C₈ alkyl groups; R₆ is chosen from —H and C₁ to C₁₂ alkyl groups; R₇ is chosen from C₁ to C₁₂ alkyl groups; R₈ is chosen from C₂ to C₈ alkyl groups; R₉ is chosen from —H and C₁ to C₁₂ alkyl groups; and R₁₀ is chosen from C₁ to C₁₂ alkyl groups.

General Formula (III) has the structure:

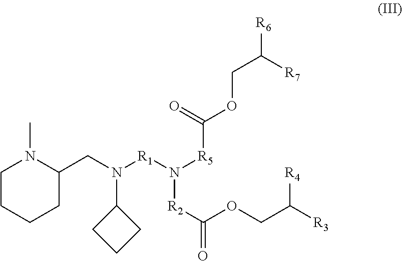

in which R₁ is chosen from C₂ to C₄ aliphatic groups, wherein the aliphatic group is selected from alkyl, alkenyl, and alkynyl groups; R₂ is chosen from C₂ to C₈ aliphatic groups, wherein the aliphatic group is selected from alkyl, alkenyl, and alkynyl groups; R₃ is chosen from C₁ to C₁₂ aliphatic groups, wherein the aliphatic group is selected from alkyl, alkenyl, and alkynyl groups; R₄ is chosen from C₁ to C₁₂ aliphatic groups, wherein the aliphatic group is selected from alkyl, alkenyl, and alkynyl groups; R₅ is chosen from C₂ to C₈ aliphatic groups, wherein the aliphatic group is selected from alkyl, alkenyl, and alkynyl groups; R₆ is chosen from C₁ to C₂₀ aliphatic groups, wherein the aliphatic group is selected from alkyl, alkenyl, and alkynyl groups; and R₇ is chosen from C₁ to C₂₀ aliphatic groups, aliphatic groups, wherein the aliphatic group is selected from alkyl, alkenyl, and alkynyl groups. In some embodiments, each of R₁-R₇ has an alkyl aliphatic group. In some embodiments, the aliphatic group of each of R₁-R₇ is independently selected.

In some embodiments, ionizable lipids of the present disclosure have the structure of General Formula (III), wherein R₁ is chosen from C₂ to C₄ alkyl groups; R₂ is chosen from C₂ to C₈ alkyl groups; R₃ is chosen from C₁ to C₁₂ alkyl groups; R₄ is chosen from C₁ to C₁₂ alkyl groups; R₅ is chosen from C₂ to C₈ alkyl groups; R₆ is chosen from C₁ to C₁₂ alkyl groups; and R₇ is chosen from C₁ to C₁₂ alkyl groups.

General Formula (IV) has the structure:

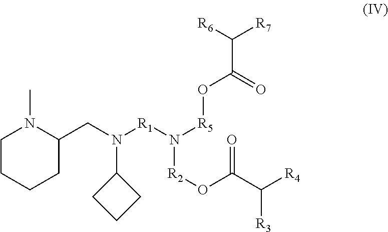

in which R₁ is chosen from C₂ to C₄ aliphatic groups, wherein the aliphatic group is selected from alkyl, alkenyl, and alkynyl groups; R₂ is chosen from C₂ to C₈ aliphatic groups, wherein the aliphatic group is selected from alkyl, alkenyl, and alkynyl groups; R₃ is chosen from —H and C₁ to C₁₂ aliphatic groups, wherein the aliphatic group is selected from alkyl, alkenyl, and alkynyl groups; R₄ is chosen from C₁ to C₁₂ aliphatic groups, wherein the aliphatic group is selected from alkyl, alkenyl, and alkynyl groups; R₅ is chosen from C₂ to C₈ aliphatic groups, wherein the aliphatic group is selected from alkyl, alkenyl, and alkynyl groups; R₆ is chosen from —H and C₁ to C₂₀ aliphatic groups, wherein the aliphatic group is selected from alkyl, alkenyl, and alkynyl groups; and R₇ is chosen from C₁ to C₂₀ aliphatic groups, aliphatic groups, wherein the aliphatic group is selected from alkyl, alkenyl, and alkynyl groups. In some embodiments, each of R₁-R₇ has an alkyl aliphatic group. In some embodiments, each of R₁-R₂, R₄-R₅ and R₇ have an alkyl aliphatic group. In some embodiments, the aliphatic group of each of R₁-R₁₀ is independently selected.

In some embodiments, ionizable lipids of the present disclosure have the structure of General Formula (IV), wherein R₁ is chosen from C₂ to C₄ alkyl groups; R₂ is chosen from C₂ to C₈ alkyl groups; R₃ is chosen from —H and C₁ to C₁₂ alkyl groups; R₄ is chosen from C₁ to C₁₂ alkyl groups; R₅ is chosen from C₂ to C₈ alkyl groups; R₆ is chosen from —H and C₁ to C₁₂ alkyl groups; and R₇ is chosen from C₁ to C₁₂ alkyl groups.

According to some embodiments discussed herein, the ionizable lipid is selected from any one of the lipids in Table 1 or a pharmaceutically acceptable salt thereof.

TABLE 1

| Lipid Name | Structure |
|---|---|
| 2-hexyldecyl 6-[(2- {cyclobutyl[2- (dimethylamino)ethyl]amino} ethyl)({6-[(2-hexyldecyl)oxy]- 6-oxohexyl})amino]hexanoate | 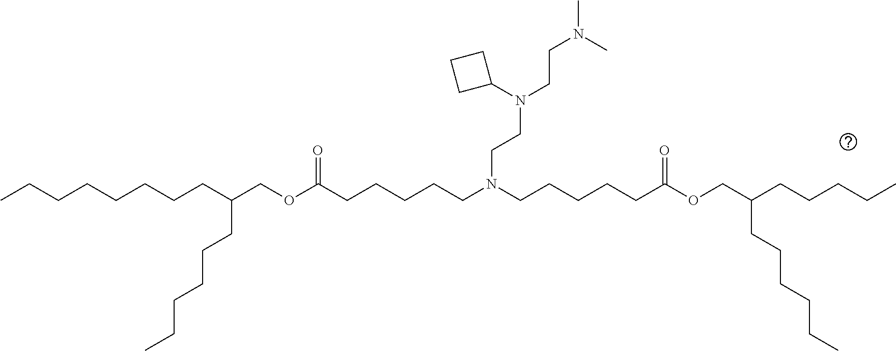 [SMILES: CCCCCCCCC(CCCCCC)COC(=O)CCCCCN(CCCCCC(=O)OCC(CCCCC)CCCCCC)CCN(CCN(C)C)C1CCC1] |
| 2-hexyldecyl 6-[(2- {cyclobutyl[3- (dimethylamino)propyl]amino} ethyl)({6-[(2-hexyldecyl)oxy]- 6-oxohexyl})amino]hexanoate | 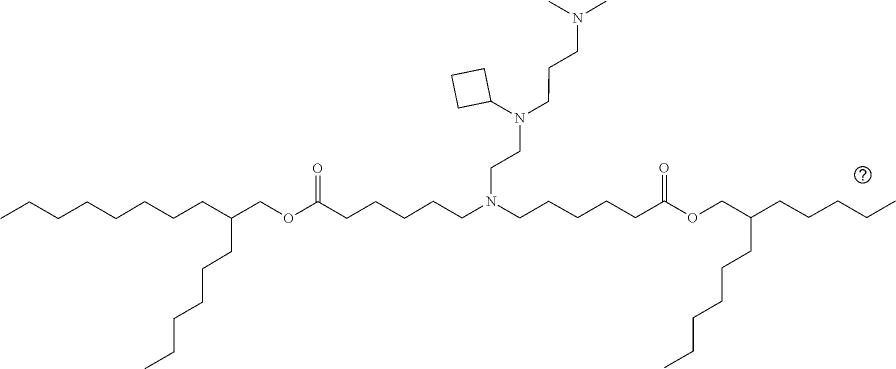 [SMILES: CCCCCCCCC(CCCCCC)COC(=O)CCCCCN(CCCCCC(=O)OCC(CCCCC)CCCCCC)CCN(CCCN(C)C)C1CCC1] |
| 2-hexyldecyl 6-[(2- {cyclobutyl[4- (dimethylamino)butyl]amino} ethyl)({6-[(2-hexyldecyl)oxy]- 6-oxohexyl})amino]hexanoate | 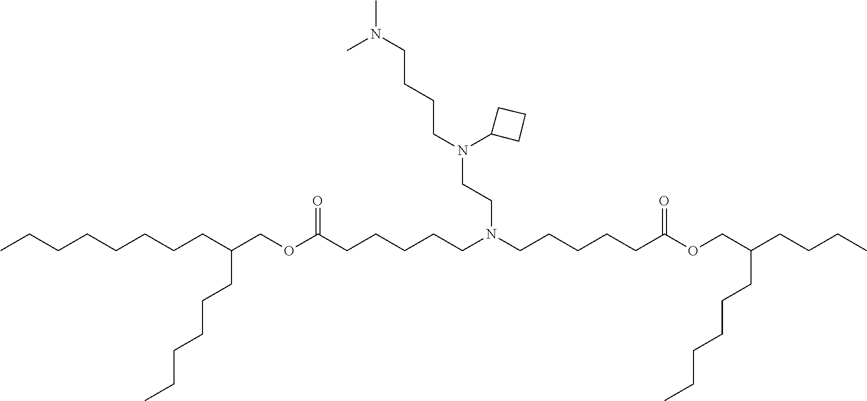 [SMILES: CCCCCCCCC(CCCCCC)COC(=O)CCCCCN(CCCCCC(=O)OCC(CCCC)CCCCCC)CCN(CCCCN(C)C)C1CCC1] |
| 2-hexyldecyl 6-[(2- {cyclobutyl[(1-methylpiperidin- 2-yl)methyl]amino}ethyl)({6- [(2-hexyldecyl)oxy]-6- oxohexyl})amino]hexanoate | 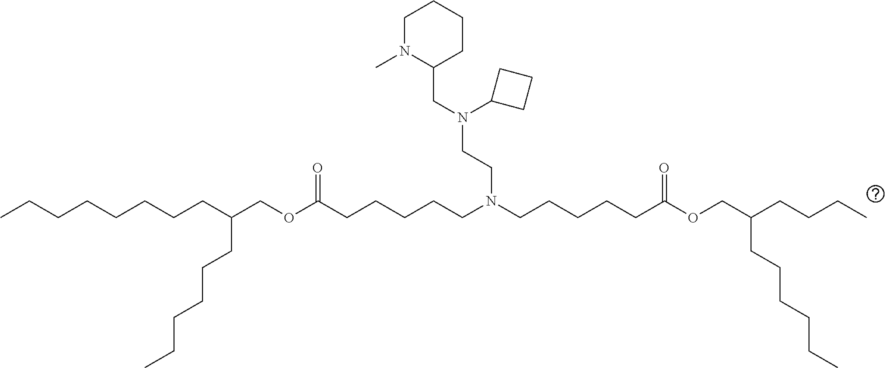 [SMILES: CCCCCCCCC(CCCCCC)COC(=O)CCCCCN(CCCCCC(=O)OCC(CCCC)CCCCCC)CCN(CC1CCCCN1C)C1CCC1] |
| 2-hexyldecyl 7-[(2- {cyclobutyl[2- (dimethylamino)ethyl]amino} ethyl)({7-[(2-hexyldecyl)oxy]-7- oxoheptyl})amino]heptanoate | 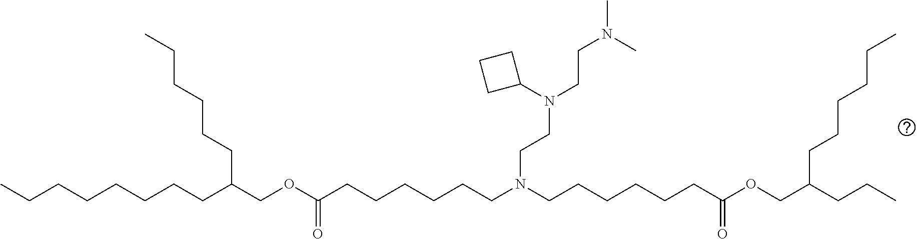 [SMILES: CCCCCCCCC(CCCCCC)COC(=O)CCCCCCN(CCCCCCC(=O)OCC(CCC)CCCCCC)CCN(CCN(C)C)C1CCC1] |
| 2-hexyldecyl 7-[(2- {cyclobutyl[3- (dimethylamino)propyl]amino} ethyl)({7-[(2-hexyldecyl)oxy]-7- oxoheptyl})amino]heptanoate | 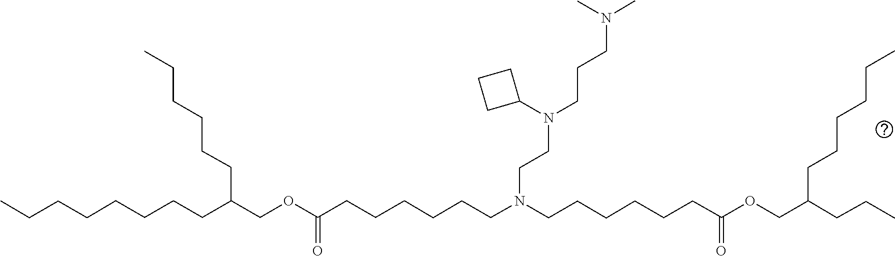 [SMILES: CCCCCCCCC(CCCCCC)COC(=O)CCCCCCN(CCCCCCC(=O)OCC(CCC)CCCCCC)CCN(CCCN(C)C)C1CCC1] |
| 2-hexyldecyl 7-[(2- {cyclobutyl[4- (dimethylamino)butyl]amino} ethyl)({7-[(2-hexyldecyl)oxy]-7- oxoheptyl})amino]heptanoate | 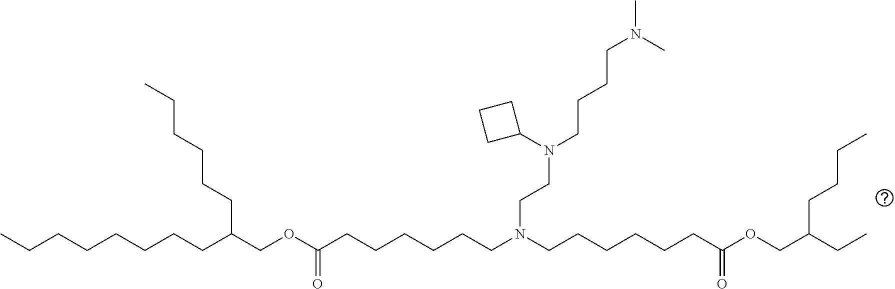 [SMILES: CCCCCCCCC(CCCCCC)COC(=O)CCCCCCN(CCCCCCC(=O)OCC(CC)CCCC)CCN(CCCCN(C)C)C1CCC1] |
| 2-hexyldecyl 6-[(3- {cyclobutyl[2- (dimethylamino)ethyl]amino} propyl)({6-[(2-hexyldecyl)oxy]- 6-oxohexyl})amino]hexanoate | 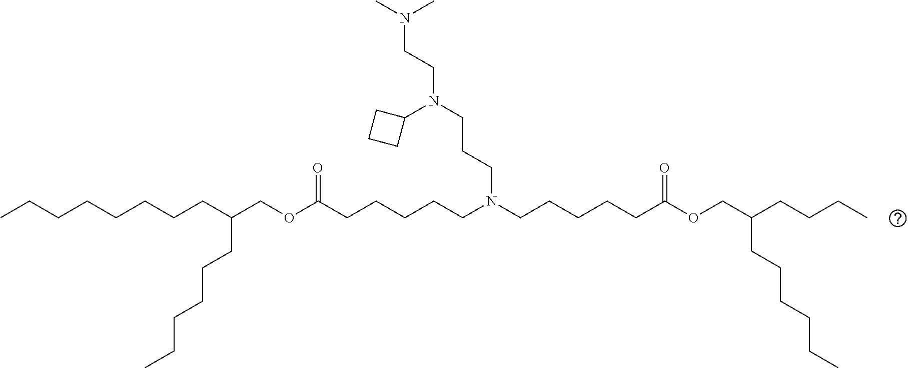 [SMILES: CCCCCCCCC(CCCCCC)COC(=O)CCCCCN(CCCCCC(=O)OCC(CCCC)CCCCCC)CCCN(CCN(C)C)C1CCC1] |
| 5-[(2-{cyclobutyl[2- (dimethylamino)ethyl]amino} ethyl)({5-[(2- hexyldecanoyl)oxy]pentyl}) amino]pentyl 2-hexyldecanoate | 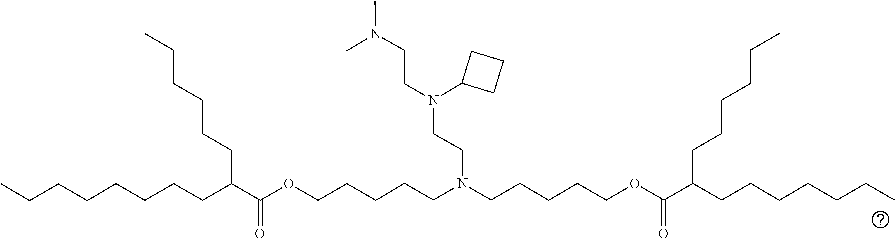 [SMILES: CCCCCCCCC(CCCCCC)C(=O)OCCCCCN(CCCCCOC(=O)C(CCCCCC)CCCCCCC)CCN(CCN(C)C)C1CCC1] |
| 5-[(2-{cyclobutyl[2- (dimethylamino)ethyl]amino} ethyl)[5- (octanoyloxy)pentyl]amino] pentyl 2-hexyldecanoate | 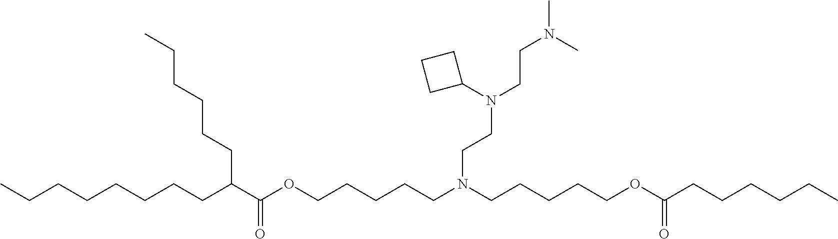 [SMILES: CCCCCCCCC(CCCCCC)C(=O)OCCCCCN(CCCCCOC(=O)CCCCCC)CCN(CCN(C)C)C1CCC1] |

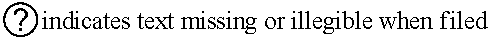

### Synthesis of Ionizable Lipids

In some embodiments, ionizable lipids of the present disclosure are synthesized by reacting an aliphatic diamine in ethanol with a cyclic ketone and a metal alkoxide to generate a first reaction product. The reaction can be performed with or without stirring. The reaction may proceed for any appropriate amount of time with or without monitoring. In some embodiments, the amount of time includes, for example, about 0-24 hours, including about 1, 2, 3, 4, 5, 6, 7, 8, 9, 10, 11, 12, 13, 14, 15, 16, 17, 18, 19, 20, 21, 22, 23, and 24 hours, or any range having endpoints defined by any two of the aforementioned values.

Illustrative aliphatic diamines include, but are not limited to, ethylenediamine, 1,1-dimethylethylenediamine, 1,2-dimethylethylenediamine, ethambutol, tetrakis(dimethylamino) ethylene, tetramethylethylenediamine, 1,3-diaminopropane (propane-1,3-diamine), butane-1,4-diamine, pentane-1,5-diamine, hexamethylenediamine (hexane-1,6 diamine), trimethyl hexamethylenediamine, 1,2-diaminopropane, trimethylenediamine, tetramethylenediamine, pentamethylenediamine, hexamethylenediamine, heptamethylenediamine, octamethylenediamine, nonamethylenediamine, decamethylenediamine, undecamethylenediamine, dodecamethylenediamine, 4,4′-diamino(dicyclohexylmethane), xylylenediamine, 1,2-dimethylethylenediamine, 1,1-dimethylethylenediamine, and the like, though any suitable aliphatic diamine is contemplated and possible.

In some embodiments, the aliphatic diamine is dimethylaminopropylamine (3-(dimethylamino)-1-propylamine). In other embodiments, the aliphatic diamine is N,N-dimethylethylenediamine. In still other embodiments, the aliphatic diamine is (4-aminobutyl) dimethylamine.

Illustrative cyclic ketones include, but are not limited to, cyclobutanone, cyclopropanone, cyclohexanone, isophorone, cyclopentanone, cycloheptanone, cyclododecanone, cyclohexadecanone, cyclooctanone, and the like, though any cyclic ketone is contemplated and possible. In some embodiments, the cyclic ketone is cyclobutanone.

Illustrative metal alkoxides include, but are not limited to, transition metal alkoxides and alkali alkoxides. In some embodiments, a transition metal alkoxide includes Co, Ga, Ge, Hf, Fe, Ni, Nb, Mo, La, Re, Sc, Si, Ti, Ta, W, Y, Zr, and the like. In some embodiments, a transition metal alkoxide includes isopropoxide, ethoxide, tert-butoxide, and the like. In some embodiments, the metal alkoxide is titanium (IV) isopropoxide.

In some embodiments, generating the first reaction product further comprises adding a reducing agent. The reducing agent can be added with or without stirring. The reducing agent can also be added to the reaction mixture after the reaction has proceeded for the appropriate amount of time. In some embodiments, the reducing agent is reacted for an additional period of time. In some embodiments, the period of time includes, for example, about 0-10 hours, including about 1, 2, 3, 4, 5, 6, 7, 8, 9, and 10 hours, or any range having endpoints defined by any two of the aforementioned values. In some embodiments, after the period of time has passed, the reaction may be quenched using any appropriate solvent, such as water. Illustrative examples of suitable reducing agents include, but are not limited to, sodium borohydride, lithium aluminum hydride, zinc amalgam, nascent hydrogen, diborane, sodium amalgam, thiosulfates, and the like, the other reducing agents are contemplated and possible. In some embodiments, the reducing agent is sodium borohydride.

In some embodiments, the first reaction product is extracted using a solvent. Illustrative solvents include, but are not limited to, polar aprotic solvents (e.g., dichloromethane (DCM), dimethyl sulfoxide (DMSO), ethyl acetate, acetone, dimethylformamide (DMF), acetonitrile, nitromethane, propylene carbonate, etc.), nonpolar hydrocarbon solvents (e.g., pentane, hexane, benzene, heptane, toluene, etc.), nonpolar ether solvents (e.g., diethyl ether, tetrahydrofuran, etc.), nonpolar chlorocarbon solvents (e.g., chloroform, etc.), polar protic solvents (e.g., ammonia, formic acid, n-butanol, isopropyl alcohol, n-propanol, ethanol, methanol, acetic acid, water etc.), or combinations thereof. In some embodiments, the solvent used for extracting the first reaction product is ethyl acetate.

Monitoring of the reaction may include, for example, thin-layer chromatography, Fourier-transform infrared spectroscopy (FTIR), Ultraviolet-visible spectroscopy (UV-Vis), nuclear magnetic resonance (NMR), temperature monitoring, pH monitoring, and the like, though any method of monitoring known in the art is contemplated and possible.

In some embodiments, the first reaction product is dried or concentrated after extraction. In embodiments, drying and/or concentrating of the product is effected by any acceptable method, including, but not limited to, evaporation at ambient temperature, use of a heat source (e.g., a steam bath, hot plate, sand bath, oven, etc.), rotary evaporation, vacuum evaporation, or gas blow-down. In some embodiments, the first reaction product is purified using column chromatography.

In embodiments, the first reaction product is reacted with a synthesized compound, discussed in further detail herein. In some embodiments, the synthesized compound is Product B (1,1′-bis(2-hexyldecyl) 6,6′-[(2-chloroethyl) imino]bis[hexanoate]). In some embodiments, the synthesized compound is dissolved in one or more solvents. In some embodiments, the solvent is ethanol.

In embodiments, a non-nucleophilic base, such as N,N-diisopropylethylamine (DIPEA) and an inorganic molecule, such as potassium iodide, are added to the solution. The reaction can be performed with or without stirring. The reaction can be performed below, at, or above ambient temperature. In some embodiments, the reaction occurs at 75° C. The reaction may proceed for any appropriate amount of time with or without monitoring. In some embodiments, the reaction occurs for about 6-24 hours, including about 6, 7, 8, 9, 20, 11, 12, 13, 14, 15, 16, 17, 18, 19, 20, 21, 22, 23, and 24 hours, or any range having endpoints defined by any two of the aforementioned values. In some embodiments, the reaction proceeds for about 16 hours. In embodiments the mixture is concentrated and one or more solvents are subsequently added. For example, in some embodiments, the mixture is concentrated in vacuo and ethyl acetate is added.

In embodiments, the mixture is extracted after the reaction has proceeded. In some embodiments, the mixture is extracted using saturated sodium thiosulfate, sodium bicarbonate and/or brine. In some embodiment, after extraction, the organic layers are separated, dried, and/or purified to yield a lipid. In some embodiments, the lipid has a structure of General Formula (I), General Formula (II), General Formula (III), and/or General Formula (IV). In some embodiments, the lipid has a structure of one of the lipids in Table 1.

### Synthesized Compound

The synthesized compound is generated by reacting a carboxylic acid with a fatty alcohol to form a reaction product. In embodiments, this reaction is performed with stirring. In other embodiments, it is performed without stirring. In embodiments, this reaction can occur at temperatures above ambient temperature, including, but not limited to, about 30, 35, 40, 45, 50, 55, 60, 65, 70, 75, 80, 85, 90, 95, or 100° C. In embodiments, this reaction can occur at about ambient temperature. In aspects, this reaction can occur below ambient temperature, including, but not limited to, about 20, 15, 10, 5, or 0° C. The reaction may proceed for any appropriate amount of time with or without monitoring. In some embodiments, the reaction occurs for about 6-24 hours, including about 6, 7, 8, 9, 20, 11, 12, 13, 14,15, 16, 17, 18, 19, 20, 21, 22, 23, and 24 hours, or any range having endpoints defined by any two of the aforementioned values. In some embodiments, the reaction proceeds for about 16 hours.

In embodiments, a carboxyl activating agent, such as 1-ethyl-3-(3-dimethylaminopropyl)carbodiimide (EDC) or N, N′-dicyclohexyl carbodiimide (DCC), and an esterification catalyst, such as 4-dimethylaminopyridine (DMAP) are added to the solution in one or more solvents. In embodiments, the solvent is dichloromethane (DCM).

Illustrative examples of carboxylic acids include, but are not limited to branched and unbranched carboxylic acids including ethanoic acids, propionic acids, butanoic acids, pentanoic acid, hexanoic acid, heptanoic acid, octonoic acid and the like. In some embodiments, the carboxylic acid is 6-bromo hexanoic acid.

Illustrative fatty alcohols include, but are not limited to, branched and unbranched fatty alcohols, including oleyl alcohol, linoleyl alcohol, tert-butyl alcohol, tert-amyl alcohol, enanthic alcohol, capryl alcohol, pelargoinc alcohol, capric alcohol, undecyl alcohol, lauryl alcohol, tridecyl alcohol, myristyl alcohol, pentadecyl alcohol, cetyl alcohol, palmitoleyl alcohol, heptadecyl alcohol, stearyl alcohol, nonadecyl alcohol, arachidyl alcohol, heneicosyl alcohol, behenyl alcohol, erucyl alcohol, lignoceryl alcohol, and ceryl alcohol, though any suitable fatty alcohol is contemplated and possible. In some embodiments, the fatty alcohol is 2-hexyl-1-decanol.

One or more solvents are added to extract the reaction product. In embodiments, the solvents are sodium bicarbonate and saturated brine. In embodiments, the reaction product is concentrated and purified. In some embodiments, the reaction product is Product A, having the structure:

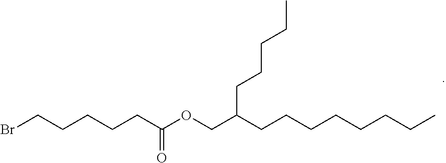 [SMILES: CCCCCCCCC(CCCCC)COC(=O)CCCCCBr ; "."]

In embodiments, the reaction product is used in the synthesis of the synthesized compound. The reaction product can be dissolved in a solution of one or more solvents. In embodiments, the solvent is ethanol. In embodiments, the reaction product is mixed with an amino alcohol. In some embodiments, the amino alcohol is 2-aminoethanol, though any amino alcohol is contemplated and possible. In embodiments, one or more inorganic compounds are added following the addition of the amino alcohol. In embodiments, the one or more inorganic compounds include potassium carbonate, cesium carbonate and/or potassium iodide. In some embodiments, potassium carbonate, cesium carbonate and potassium iodide are added. The reaction can be performed with or without stirring. The reaction can be performed below, at, or above ambient temperature. In some embodiments, the reaction occurs at 75° C. The reaction may proceed for any appropriate amount of time with or without monitoring. In some embodiments, the amount of time ranges from about 24-48 hours, including about 24, 25, 26, 27, 28, 29, 30, 31, 32, 33, 34, 35, 36, 37, 38, 39, 40, 41, 42, 43, 44, 45, 46, 47, or 48, or any range having endpoints defined by any two of the aforementioned values. In some embodiments, the reaction proceeds for about 48 hours. In some embodiments, the mixture is extracted, concentrated, dried and/or purified to yield a second reaction product.

In some embodiments, the second reaction product is 1′-Bis(2-hexyldecyl) 6,6′-[(2-hydroxyethyl)imino]bis[hexanoate], having the structure:

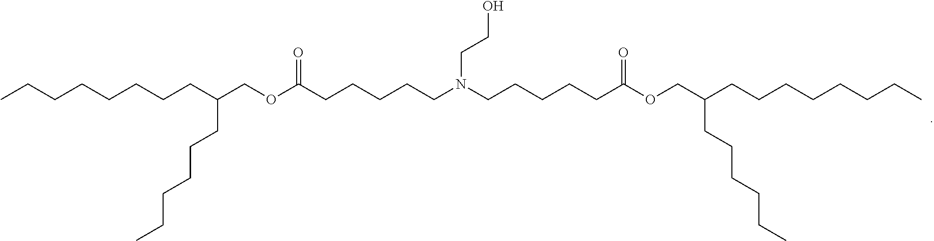 [SMILES: CCCCCCCCC(CCCCCC)COC(=O)CCCCCN(CCO)CCCCCC(=O)OCC(CCCCCC)CCCCCCCC]

In embodiments, the second reaction product is dissolved in a solvent. In some embodiments, the solvent is chloroform. In embodiments, an inorganic compound is added to the mixture. In some embodiments, the inorganic compound is thionyl chloride. In some embodiments, the inorganic compound is added dropwise. The reaction can be performed with or without stirring. The reaction can be performed below, at, or above ambient temperature. In some embodiments, the reaction occurs at ambient temperature. The reaction may proceed for any appropriate amount of time with or without monitoring. In some embodiments, the amount of time ranges from about 24-48 hours, including about 24, 25, 26, 27, 28, 29, 30, 31, 32, 33, 34, 35, 36, 37, 38, 39, 40, 41, 42, 43, 44, 45, 46, 47, or 48, or any range having endpoints defined by any two of the aforementioned values. In some embodiments, the reaction proceeds for about 48 hours. In some embodiments, the mixture is extracted, concentrated, dried and/or purified to yield the synthesized compound.

In some embodiments, the synthesized compound is Product B (1,1′-bis(2-hexyldecyl) 6,6′-[(2-chloroethyl) imino]bis[hexanoate]), having the structure:

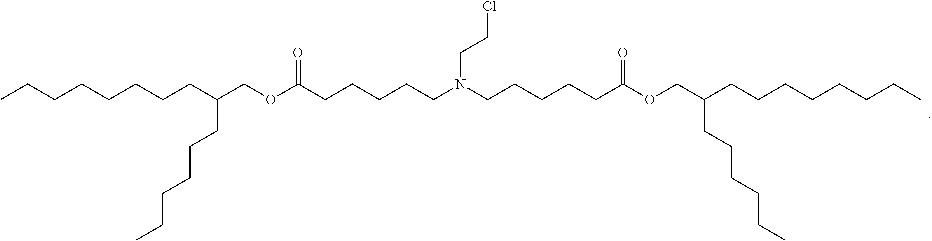 [SMILES: CCCCCCCCC(CCCCCC)COC(=O)CCCCCN(CCCl)CCCCCC(=O)OCC(CCCCCC)CCCCCCCC]

### Lipid Nanoparticles

As used herein, the term “nanoparticle” refers to a particle having dimensions on a scale of less than about 1000 nm. Routinely, nanoparticles have any one structural feature on a scale of less than about 500 nm, less than about 400 nm, less than about 300 nm, less than about 200 nm or less than about 100 nm. In exemplary embodiments, a nanoparticle is a particle having one or more dimensions of the order of about 10-500 nm. In other exemplary embodiments, a nanoparticle is a particle having one or more dimensions of the order of about 10-1000 nm. A spherical nanoparticle would have a diameter, for example, of between 10-100 nm or 10-1000 nm.

The term “particle size” or “particle diameter” refers to the mean diameter of the particles in a sample, as measured by dynamic light scattering (DLS), multiangle light scattering (MALS), nanoparticle tracking analysis, or comparable techniques. It will be understood that a dispersion of lipid nanoparticles as described herein will not be of uniform size but can be described by the average diameter and, optionally, the polydispersity index.

In some embodiments of this disclosure, the ionizable lipids disclosed herein, having any of General Formula (I), General Formula (II), General Formula (III), and/or General Formula (IV) particularly those identified in Table 1 may be incorporated into lipid nanoparticles (LNPs). In some aspects, the lipid nanoparticles may be used to deliver cargo molecules (e.g. polypeptides, nucleic acids, small molecules, etc.) alone or as packaged in a deliverable pharmaceutical composition, such as a vaccine. Lipid nanoparticles may include one or more ionizable lipids, one or more cationic lipids, one or more helper lipids, one or more structural lipids or sterols and/or one or more polymer-conjugated lipids along with nucleic acid or polypeptide cargo of interest.

LNPs may be used in some aspects to carry and/or deliver cargo to a subject or a portion thereof such as a cell or cellular compartment. DNA and RNA vaccines utilizing such LNPs share many similarities, but each targets different cellular environments. For example, DNA vaccines target and are used in the nucleus of a cell, whereas RNA vaccines target and are expressed in the cytosol. This makes mRNA vaccines easier to deliver, yet both may capitalize on the success of recent advances in LNP formulations and sometimes other modifications to the nucleic acid cargo itself that may improve overall function.

In some embodiments, lipid nanoparticles described herein can have an average particle diameter that is about 30 nm, 35 nm, 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185, nm, 190 nm, 195 nm, 200 nm, 205 nm, 210 nm, 215 nm, 220 nm, 225 nm, 230 nm, 235 nm, 240 nm, 245 nm, 250 nm, 255 nm, 260 nm, 265 nm, 270 nm, 275 nm, 280 nm, 285, nm, 290 nm, 295 nm, 300 nm, 305 nm, 310 nm, 315 nm, 320 nm, 325 nm, 330 nm, 335 nm, 340 nm, 345 nm, 350 nm, 355 nm, 360 nm, 365 nm, 370 nm, 375 nm, 380 nm, 385, nm, 390 nm, 395 nm, 400 nm, 405 nm, 410 nm, 415 nm, 420 nm, 425 nm, 430 nm, 435 nm, 440 nm, 445 nm, 450 nm, 455 nm, 460 nm, 465 nm, 470 nm, 475 nm, 480 nm, 485, nm, 490 nm, 495 nm, 500 nm, or any range having endpoints defined by any two of the aforementioned values. For example, in some embodiments, lipid nanoparticles described herein have an average particle diameter from between 100 nm to 200 nm.

The zeta potential of a nanoparticle composition may be used to indicate the electrokinetic potential of the composition. For example, the zeta potential may describe the surface charge of a nanoparticle composition. Nanoparticle compositions with relatively low charges, positive or negative, are generally desirable, as more highly charged species may interact undesirably with cells, tissues, and other elements in the body. In some embodiments, the zeta potential of a nanoparticle composition may be from about −20 mV to about +20 mV, from about −20 mV to about +15 mV, from about −20 mV to about +10 mV, from about −20 mV to about +5 mV, from about −20 mV to about 0 mV, from about −20 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

In some embodiments, the lipid nanoparticles comprise one or more ionizable lipids having any of General Formula (I), General Formula (II), General Formula (III), and/or General Formula (IV) as described herein. In some embodiments, ionizable lipids constitute at least about 5%, 10%, 20%, 30%, 35%, 40%, 45%, 50%, 55%, 60%, 65%, 70%, or 75% of the total lipids in a suitable lipid solution by weight or by molar. In some embodiments, ionizable lipids constitute about 30-70% (e.g., about 30-65%, about 30-60%, about 30-55%, about 30-50%, about 30-45%, about 30-40%, about 35-50%, about 35-4 5%, about 35-40%, about 40-60%, about 45-60%, about 50-60%, about 55-60, about 40-65%, about 45-65%, about 50-65%, about 55-65%, about 60-65%) of the total lipid nanoparticle composition by weight or by molar.

In some embodiments, the lipid nanoparticles comprise one or more cationic lipids in combination with the ionizable lipids. In some embodiments, the lipid nanoparticles comprise one or more helper lipids in combination with the ionizable lipids and/or the cationic lipids. In some embodiments, the lipid nanoparticles comprise one or more structural lipids or sterols in combination with the ionizable lipids and/or the cationic lipids and/or the helper lipids. In some embodiments, the lipid nanoparticles comprise one or more polymer-conjugated lipids in combination with the ionizable lipids and/or the cationic lipids and/or the helper lipids and/or the structural lipids or sterols.

### Cationic Lipids

In some embodiments, lipid nanoparticles of the present disclosure contain one or more cationic lipids in combination with any of the above ionizable lipids for the formation of lipid nanoparticles. Suitable cationic lipids include, but are not limited to 3-(didodecylamino)-NI,N1,4-tridodecyI-1-piperazineethanamine (KL10), NI-[2-(didodecylamino)ethyl]-NI,N4,N4-tridodecyI-1,4-piperazinediethanamine (KL22), 14,25-ditridecyl-15,1 8,21,24-tetraaza-octatriacontane (KL25), 2,2-dilinoleyl-4-dimethylaminomethyl-[1,3]-dioxolane (DLin-K-DMA), 2-({8-[(3P)-cholest-5-en-3-yloxy]octyl}oxy)-N,N-dimethyl-3-[(9Z,12Z)-octadeca-9,12-dien-1-yloxy]propan-1-amine (Octyl-CLinDMA), N-[1-(2,3-dioleyloxy)propyl]-N,N,N-trimethylammonium chloride (DOTMA), 5-carboxyspermylglycinedioctadecylamide (DOGS) 2,3-dioleyloxy-N-[2(spermine-carboxamido)ethyl]-N,N-dimethyl-1-propanaminium (DOSPA), 1,2-dioleoyl-3-dimethylammonium-Propane (DODAP), 1,2-dioleoyl-3-trimethylammonium-propane (DOTAP), 1,2-distearyloxy-N,N-dimethyl-3-aminopropane (DSDMA), 1,2-dioleyloxy-N,N-dimethyl-3-aminopropane (DODMA), 1,2-dilinoleyloxy-N,N-dimethyl-3-aminopropane (DLinDMA), 1,2-dilinolenyloxy-N,N-dimethyl-3-aminopropane (DLenDMA), N-dioleyl-N,N-dimethylammonium chloride (DODAC), N,N-distearyl-N,N-dimethylarnrnonium bromide (DDAB), N-(1,2-dimyristyloxyprop-3-yl)-N,N-dimethyl-N-hydroxyethyl ammonium bromide (DMRIE), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′, 1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 2,3-Dilinoleoyloxy-N,N-dimethylpropylamine (DLinDAP), 1,2-N,N′-Dilinoleylcarbamyl-3-dimethylaminopropane (DLincarbDAP), 1,2-Dilinoleoylcarbamyl-3-dimethylaminopropane (DLinCDAP), 2,2-dilinoleyl-4-dimethylaminomethyl-[1,3]-dioxolane (DLin-DMA), 2,2-dilinoleyl-4-dimethylaminoethyl-[1,3]-dioxolane (DLin-K-XTC2-DMA), 2-(2,2-di((9Z,12Z)-octadeca-9,1 2-dien-1-yl)-1,3-dioxolan-4-yl)-N,N-dimethylethanamine (DLin-KC2-DMA), heptatriaconta-6,9,28,31-tetraen-19-yl 4-(dimethylamino)butanoate (DLin-MC3-DMA), (6Z,9Z,28Z,31Z)-heptatriaconta-6,9,28,31-tetraen-19-yl4-(dimethylamino) butanoate (MC3), and the like, and combinations thereof.

In some embodiments, the lipid nanoparticles include one or more cationic lipids selected from the group consisting of 3-(didodecylamino)-NI,N1,4-tridodecyI-1-piperazineethanamine (KL10), NI-[2-(didodecylamino)ethyl]-NI,N4,N4-tridodecyI-1,4-piperazinediethanamine (KL22), 14,25-ditridecyl-15,1 8,21,24-tetraaza-octatriacontane (KL25), 1.2-dilinoleyloxy-N,N-dimethylaminopropane (DLin-DMA), 2.2-dilinoleyl-4-dimethylaminomethyl-[1,3]-dioxolane (DLin-K-DMA), heptatriaconta-6,9,28,31-tetraen-19-yl 4-(dimethylamino)butanoate (DLin-MC3-DMA), 2.2-dilinoleyl-4-(2-dimethylaminoethyl)-[1,3]-dioxolane (DLin-KC2-DMA), 1.2-dioleyloxy-N,N-dimethylaminopropane (DODMA), 2-({8-[(3P)-cholest-5-en-3-yloxy]octyl}oxy)-N,N-dimethyl-3-[(9Z,12Z)-octadeca-9,12-dien-1-yloxy]propan-1-amine (Octyl-CLinDMA), and combinations thereof.

In some embodiments, cationic lipids constitute at least about 5%, 10%, 20%, 30%, 35%, 40%, 45%, 50%, 55%, 60%, 65%, 70%, or 75% of the total lipids in a suitable lipid solution by weight or by molar. In some embodiments, cationic lipids constitute about 30-70% (e.g., about 30-65%, about 30-60%, about 30-55%, about 30-50%, about 30-45%, about 30-40%, about 35-50%, about 35-45%, about 35-40%, about 40-60%, about 45-60%, about 50-60%, about 55-60, about 40-65%, about 45-65%, about 50-65%, about 55-65%, about 60-65%) of the total lipid nanoparticle composition by weight or by molar.

### Helper Lipids

In embodiments, the liquid nanoparticles can further comprise one or more non-cationic, helper lipids. The helper lipid can serve to increase fusogenicity and/or increase stability of the lipid nanoparticle during formation. Helper lipids can include, but are not limited to, phospholipids, neutral lipids and anionic lipids. As used herein, the phrase “helper lipid” refers to any neutral, zwitterionic or anionic lipid.

Helper lipids include, but are not limited to, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoylphosphatidylethanolamine (DOPE), 2-diphytanoyl-sn-glycero-3-phosphatidylethanolamine (DPyPE), distearoyl-sn-glycero-phosphoethanolamine, palmitoyloleoylphosphatidylcholine (POPC), palmitoyloleoyl-phosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), egg sphingomyelin (ESM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), 1,2-dilauroyl-sn-glycero-3-phosphoethanolamine (DLPE); 1,2-diphytanoyl-sn-glycero-3-phosphoethanolamine (DPHyPE); lecithin, phosphatidylethanolamine, lysolecithin, lysophosphatidylethanolamine, phosphatidylserine, phosphatidylinositol, and the like, and combinations thereof.

As used herein, the term “neutral lipid” is meant to refer to any of a number of lipid species that exist either in an uncharged or neutral zwitterionic form at a selected pH. At physiological pH, such lipids include, but are not limited to, for example, diacylphosphatidylcholine, diacylphosphatidylethanolamine, ceramide, sphingomyelin, cephalin, cholesterol, cerebrosides, and diacylglycerols.

Additional exemplary neutral lipids include, for example, dipalmitoylphosphatidylglycerol (DPPG), palmitoyloleoyl-phosphatidylethanolamine (POPE) and dioleoyl-phosphatidylethanolamine 4-(N-maleimidomethyl)-cyclohexane-carboxylate (DOPE-mal), dipalmitoyl phosphatidyl ethanolamine (DPPE), dimyristoylphosphoethanolamine (DMPE), distearoyl-phosphatidylethanolamine (DSPE), 16-O-monomethyl PE, 16-O-dimethyl PE, 18-1-trans PE, 1-stearioyl-2-oleoylphosphatidyethanol amine (SOPE), and 1,2-dielaidoyl-sn-glycero-3-phophoethanolamine (transDOPE) and combinations thereof. In some embodiments, the neutral lipid is 1,2-distearoyl-sn-glycero-3phosphocholine (DSPC). In some embodiments, the neutral lipid is selected from DSPC, DPPC, DMPC, DOPC, POPC, DOPE, and SM.

In some embodiments, the helper lipid includes one or more anionic lipids. As used herein, the term “anionic lipid” refers to any lipid that is negatively charged at physiological pH. These lipids include, but are not limited to, phosphatidylglycerols, cardiolipins, diacylphosphatidylserines, diacylphosphatidic acids, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), and other anionic modifying groups joined to neutral lipids, and combinations thereof.

In some embodiments, the helper lipid comprises one or more phospholipids, such as one or more (poly) unsaturated lipids. Without being bound by the theory, it is contemplated that phospholipids may assemble into one or more lipid bilayers structures.

Exemplary phospholipids that can form part of the present nanoparticle composition include but are not limited to 1, 2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1, 2-dioleoyl-sn-glycero-3-phospho-rac-(1-glycerol) sodium salt (DOPG), and sphingomyelin. In certain embodiments, a nanoparticle composition includes DSPC. In certain embodiments, a nanoparticle composition includes DOPE. In some embodiments, a nanoparticle composition includes both DSPC and DOPE.

In some embodiments, the helper lipid is phosphatidylcholine (PC), phosphatidylethanolamine (PE) phosphatidylserine (PS), phosphatidic acid (PA), or phosphatidylglycerol (PG).

Additionally, phospholipids that can form part of the present nanoparticle composition also include those described in WO2017/112865, the entire content of which is hereby incorporated by reference.

In some embodiments, helper lipids may constitute at least about 5%, 10%, 15%, 20%, 25%, 30%, 35%, 40%, 45%, or 50% of the total lipids in a suitable lipid solution by weight or by molar. In some embodiments, helper lipid(s) constitute(s) about 5-25% (e.g., about 5-20%, about 5-15%, about 5-10%, about 10-25%, about 10-20%, about 10-15%, about 15-25%, about 15-20%, or about 20-25%) of the total lipids in a suitable lipid solution by weight or by molar.

### Structural Lipids/Sterols

In some embodiments, the lipid nanoparticles further comprise a structural lipid or sterol. Without being bound by theory, it is contemplated that structural lipids and/or sterols can stabilize the amphiphilic structure of a nanoparticle to provide membrane integrity and stability of the lipid particle. Illustrative examples of structural lipids and sterols include, but are not limited to, cholesterol, ergosterol, campesterol, oxysterol, antrosterol, desmosterol, nicasterol, sitosterol, stigmasterol, fecosterol, brassicasterol, tomatidine, tomatine, ursolic acid, alpha-tocopherol, and combinations thereof.

In some embodiments, the lipid nanoparticles include a sterol. In some embodiments, the sterol is cholesterol or a derivative or variant thereof. Non-limiting examples of cholesterol derivatives include 5a-cholestanol, 5P-coprostanol, cholesteryl-(2′-hydroxy)-ethyl ether, cholesteryl-(4′-hydroxy)-butyl ether, 6-ketocholestanol; 5a-cholestane, cholestenone, 5a-cholestanone, 5P-cholestanone, cholesteryl decanoate, 25-hydroxycholesterol (25-OH), 20a-hydroxycholesterol (20a-OH), 27-hydroxycholesterol, 6-keto-5a-hydroxycholesterol, 7-ketocholesterol, 7-hydroxycholesterol, 7a-hydroxycholesterol, 7-25-dihydroxycholesterol, beta-sitosterol, stigmasterol, brassicasterol, campesterol, or combinations thereof.

In some embodiments, the sterol includes cholesterol and a corticosteroid (such as prednisolone, dexamethasone, prednisone, and hydrocortisone), derivatives and variants thereof, and combinations of the foregoing.

In some embodiments, structural lipids and/or sterols constitute at least about 5%, 10%, 20%, 30%, 40%, 50%, 60%, or 70% of the total lipids in a suitable lipid solution by weight or by molar. In some embodiments, structural lipids and/or sterols constitute about 30-50% (e.g., about 30-45%, about 30-40%, about 32-40%, about 35-50%, about 35-45%, about 39-49%, about 40-46%, about 40-44%, about 40-42%, about 42-44%, about 44-46%, or about 35-40%) of the total lipids in a suitable lipid solution by weight or by molar. In some embodiments, the structural lipid and/or sterol present in a concentration of 39, 40, 41, 42, 43, 44, 45, 46, 47, 48, or 49 molar percent.

In some embodiments, the molar ratio of ionizable lipid to the structural lipid and/or sterol ranges from 1.0:0.9 to 1.0:1.2, or from 1.0:1.0 to 1.0:1.2. In some embodiments, the molar ratio of ionizable lipid to cholesterol ranges from about 5:1 to 1:1.

### Polymer-Conjugated Lipids

In some embodiments, the lipid nanoparticles may further include a polymer-conjugates lipid. As used herein, the term “polymer-conjugated lipid” is meant to refer to a conjugated lipid that inhibits aggregation of lipid nanoparticles. Without being bound by the theory, it is contemplated that a polymer conjugated lipid component in a nanoparticle composition can improve of colloidal stability and/or reduce protein absorption of the nanoparticles. Such polymer-conjugated lipids include, but are not limited to, polyethylene glycol (PEG)-lipid conjugates such as, e.g., PEG coupled to dialkyloxypropyls (e.g, PEG-DAA conjugates), PEG coupled to diacylglycerols (e.g, PEG-DAG conjugates), PEG coupled to cholesterol, PEG coupled to phosphatidylethanolamines, and PEG conjugated to ceramides, ionizable PEG lipids, PEG coupled to phosphatidic acids, PEG-modified dialkylamines, PEG-modified dialkylglycerols, polyoxazoline (POZ)-lipid conjugates, polyamide oligomers (e.g, ATTA-lipid conjugates), and mixtures thereof. PEG or POZ can be conjugated directly to the lipid or may be linked to the lipid via a linker moiety. Any linker moiety suitable for coupling the PEG or the POZ to a lipid can be used including, e.g, non-ester containing linker moieties and ester-containing linker moieties. In certain preferred embodiments, non-ester containing linker moieties, such as amides or carbamates, are used.

In some embodiments, polymer-conjugated lipids include a PEG-modified lipid. In embodiments, the PEGylated lipid can be used to enhance lipid nanoparticle colloidal stability in vitro and circulation time in vivo. Illustrative PEG-lipids for use in lipid nanoparticles include, but are not limited to PEG-c-DOMG, PEG-DMG, PEG-DLPE, PEG-DMPE, PEG-DPPE, PEG-DSG, PEG-DSPE, DiMystyrlGlycerol (DMG), 1,2-Dipalmitoyl-rac-glycerol, methoxypolyethylene Glycol (DPG-PEG), 1,2-Distearoyl-rac-glycero-3-methylpolyoxyethylene (DSG-PEG), Ceramide-PEG2000, or Chol-PEG2000. In some embodiments, a polymer-conjugate lipid has an average molecular mass from about 500 Da to about 5000 Da.

In some embodiments, the PEG-modified lipid includes a pegylated diacylglycerol (PEG-DAG) such as 1-(monomethoxy-polyethyleneglycol)-2, 3-dimyristoylglycerol (PEG-DMG), a pegylated phosphatidylethanoloamine (PEG-PE), a PEG succinate diacylglycerol (PEG-S-DAG) such as 4-O-(2′,3′-di(tetradecanoyloxy) propyl-1-O-(ω-methoxy (polyethoxy) ethyl) butanedioate (PEG-S-DMG), a pegylated ceramide (PEG-cer), or a PEG dialkoxypropylcarbamate such as ω-methoxy (polyethoxy) ethyl-N-(2,3-di(tetradecanoxy) propyl) carbamate or 2,3-di(tetradecanoxy) propyl-N-(ω-methoxy (polyethoxy) ethyl) carbamate.

Polymer-conjugated lipids may constitute at least about 1%, 2%, 3%, 4%, 5%, 6%, 7%, 8%, 9%, 10%, 15% or 20% of the total lipids in a suitable lipid solution by weight or by molar. In some embodiments, polymer-conjugated lipids constitute about 1-5% (e.g., about 1-2%, about 1-3%, about 1-4%, about 2-5%, about 2-4%, about 2-3%, about 3-5%, about 3-4%, or about 4-5%) of the total lipids in a suitable lipid solution by weight or by molar. In some embodiments, the polymer-conjugated lipid is present in a concentration ranging from about 1.0% to about 2.5% molar percent. In some embodiments, the polymer-conjugated lipid is present in a concentration of about 1.7 molar percent. In some embodiments, the polymer-conjugated lipid is present in a concentration of about 1.5 molar percent.

### Lipid Nanoparticle Solution

In some embodiments, the lipid nanoparticles are formed from a lipid solution. A suitable lipid solution may contain a mixture of desired lipids at various concentrations. For example, a suitable lipid solution may contain a mixture of desired lipids at a total concentration of or greater than about 0.1 mg/ml, 0.5 mg/ml, 1.0 mg/ml, 2.0 mg/ml, 3.0 mg/ml, 4.0 mg/ml, 5.0 mg/ml, 6.0 mg/ml, 7.0 mg/ml, 8.0 mg/ml, 9.0 mg/ml, 10 mg/ml, 15 mg/ml, 20 mg/ml, 30 mg/ml, 40 mg/ml, 50 mg/ml, or 100 mg/ml. In some embodiments, a suitable lipid solution may contain a mixture of desired lipids at a total concentration ranging from about 0.1-100 mg/ml, 0.25-50 mg/ml, 1.0-20 mg/ml, 1.0-70 mg/ml, 1.0-60 mg/ml, 1.0-50 mg/ml, 1.0-40 mg/ml, 1.0-30 mg/ml, 1.0-20 mg/ml, 1.0-15 mg/ml, 1.0-10 mg/ml, 1.0-9 mg/ml, 1.0-8 mg/ml, 1.0-7 mg/ml, 1.0-6 mg/ml, or 1.0-5 mg/ml. In some embodiments, a suitable lipid solution may contain a mixture of desired lipids at a total concentration up to about 100 mg/ml, 90 mg/ml, 80 mg/ml, 70 mg/ml, 60 mg/ml, 50 mg/ml, 40 mg/ml, 30 mg/ml, 20 mg/ml, 10 mg/ml, 5 mg/mL, 4 mg/mL, 3 mg/mL, 2 mg/mL, 1 mg/mL, 0.5 mg/mL, 0.25 mg/mL, or 0.1 mg/mL.

In some embodiments, the ionizable lipid is included in the lipid solution in a molar percentage from about 20%-60%, including about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, and 60%, or any range having endpoints defined by any two of the aforementioned values. In some embodiments, the cationic lipid is included in the lipid solution in a molar percentage from about 20%-60%, including about 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, and 60%, or any range having endpoints defined by any two of the aforementioned values. In some embodiments, the helper lipid is included in the lipid solution in a molar percentage from about 0%-30%, including 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, and 30%, or any range having endpoints defined by any two of the aforementioned values. In embodiments, the structural lipid and/or sterol is included in the lipid solution in a molar percentage from about 25%-50%, including about 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, and 50%, or any range having endpoints defined by any two of the aforementioned values. In embodiments, the polymer-conjugated lipid included in the lipid solution in a molar percentage from about 0%-10%, including 1%, 2%, 3%, 4%, 5%, 6%, 7%, 8%, 9%, and 10%, or any range having endpoints defined by any two of the aforementioned values.

In some embodiments, the cargo includes a nucleic acid (e.g., DNA, RNA, e.g., mRNA). The nucleic acid may be at a concentration between 50 μg per ml and 200 μg per ml of the aqueous solution (e.g., about 50 μg/ml, about 60 μg/ml, about 70 μg/ml, about 80 μg/ml, about 90 μg/ml, about 100 μg/ml, about 110 μg/ml, about 120 μg/ml, about 130 μg/ml, about 140 μg/ml, about 150 μg/ml, about 175 μg/ml, or about 200 μg/ml). All of or a portion of the nucleic acid may be encapsulated in the lipid nanoparticles. In some embodiments, the method yields a nucleic acid encapsulation efficiency of at least 80% (e.g., 80%, 81%, 82%, 83%, 84%, 85%, 86%, 87%, 88%, 89%, 90%, 91%, 92%, 93%, 94%, 95%, 96%, 97%, 98%, 99%, or greater). In some embodiments, the method yields a nucleic acid encapsulation efficiency of at least 90%. In some embodiments, the method yields a nucleic acid encapsulation efficiency between about 90% and about 97%.

Generally, the lipid nanoparticles are prepared at a molar ratio between the amine group of the ionizable lipid and the phosphate group of the mRNA, from about 5:1 to 60:1. In some embodiments, the lipid to nucleic acid ratio (mass/mass ratio; w/w ratio) can be in the range of from about 1:1 to about 60:1, from about 1:1 to about 20:1, from about 1:1 to about 19:1, from about 1:1 to about 18:1, from about 1:1 to about 17:1, from about 1:1 to about 16:1, from about 1:1 to about 15:1, from about 1:1 to about 14:1, from about 1:1 to about 13:1, from about 1:1 to about 12:1, from about 1:1 to about 10:1, from about 1:1 to about 9:1, from about 1:1 to about 8:1, from about 1:1 to about 7:1, from about 1:1 to about 6:1, from about 1:1 to about 5:1, from about 3:1 to about 15:1, from about 4:1 to about 15:1, from about 5:1 to about 15:1, about 6:1 to about 15:1, from about 7:1 to about 15:1, from about 8:1 to about 15:1, from about 9:1 to about 15:1, from about 5:1 to about 10:1, from about 6:1 to about 10:1, from about 7:1 to about 10:1, from about 8:1 to about 10:1, or from about 9:1 to about 10:1.

The lipid nanoparticles can be prepared with the cargo at a volume ratio with the lipid solution, such that the lipid solution:cargo ratio is from about 1:1 to 10:1, including 1:1, 2:1, 3:1, 4:1, 5:1, 6:1, 7:1, 8:1, 9:1 and 10:1, or any range having endpoints defined by any two of the aforementioned values.

### EXAMPLES

The following examples are given by way of illustration and are in no way intended to limit the scope of the present disclosure.

### Example 1: Synthesis of Control 1 and 1,1′-bis(2-hexyldecyl) 6,6′-[(2-chloroethyl)imino]bis[hexanoate]

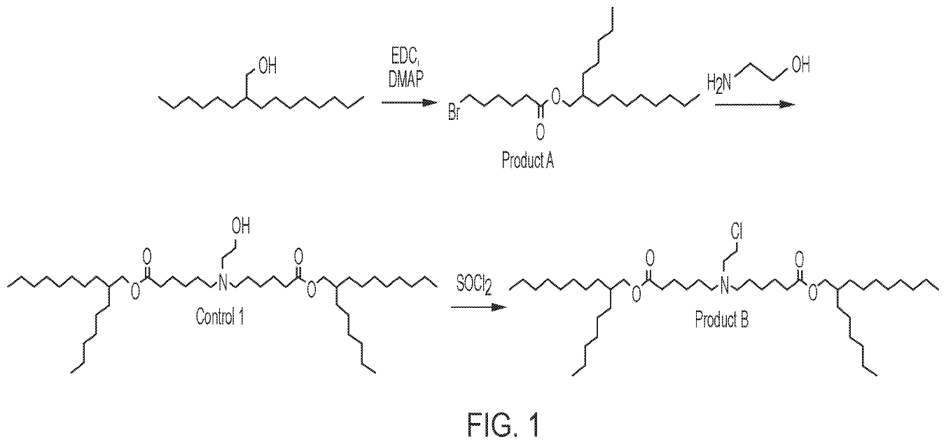

As depicted in FIG. 1, 6-bromo hexanoic acid (1.75 equiv.) and 2-hexyl-1-decanol (1 equiv.) were mixed with a solution of 1-ethyl-3-(3-dimethylaminopropyl) carbodiimide (EDC) (2.5 equiv.) and 4-dimethylaminopyridine (DMAP) (0.2 equiv.) in dichloromethane (DCM). The mixture was stirred at room temperature for sixteen hours. DCM was added to the reaction mixture. The reaction mixture was extracted using sodium bicarbonate saturated brine. The mixture was concentrated and Product A was separated using column chromatography with a gradient of ethyl acetate in hexane.

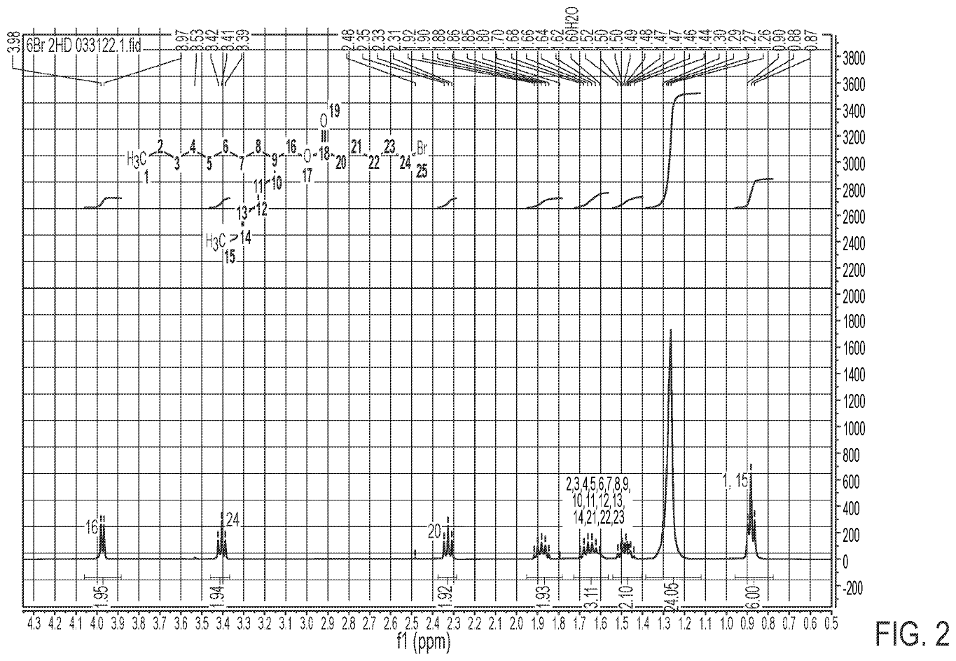

Product A was characterized by proton nuclear magnetic resonance on a 400 MHz spectrometer in CDCl₃, the results of which are depicted in FIG. 2. In particular, the data reported includes: chemical shift (6 ppm), multiplicity, integration, and coupling constant (Hz). ¹H NMR (400 MHz, CDCl₃) δ 3.97 (d, J=5.8 Hz, 2H), 3.41 (t, J=6.8 Hz, 2H), 2.33 (t, J=7.4 Hz, 2H), 1.88 (p, J=7.0 Hz, 2H), 1.66 (tt, J=15.3, 7.5 Hz, 3H), 1.48, J=10.0, 6.0 Hz, 2H), 1.27 (d, J=3.9 Hz, 24H), 0.88 (t, J=6.7 Hz, 6H).

Without further purification, Product A (2 equiv.) was added to a solution of 2-aminoethanol (1 equiv.) in ethanol. Potassium carbonate (2 equiv.), cesium carbonate (0.5 equiv.) and potassium iodide (1 equiv.) were added to the reaction mixture. The resulting mixture was stirred at 75° C. for two days. The mixture was then concentrated in vacuo. Ethyl acetate was added, and the mixture was extracted using saturated sodium bicarbonate and brine. The organic layers were dried using sodium sulfate and the resultant 1,1′-bis(2-hexyldecyl) 6,6′-[(2-hydroxyethyl)imino]bis[hexanoate](Control 1) was purified using column chromatography (0-20% (mixture of 10% NH₄OH in methanol) in dichloromethane).

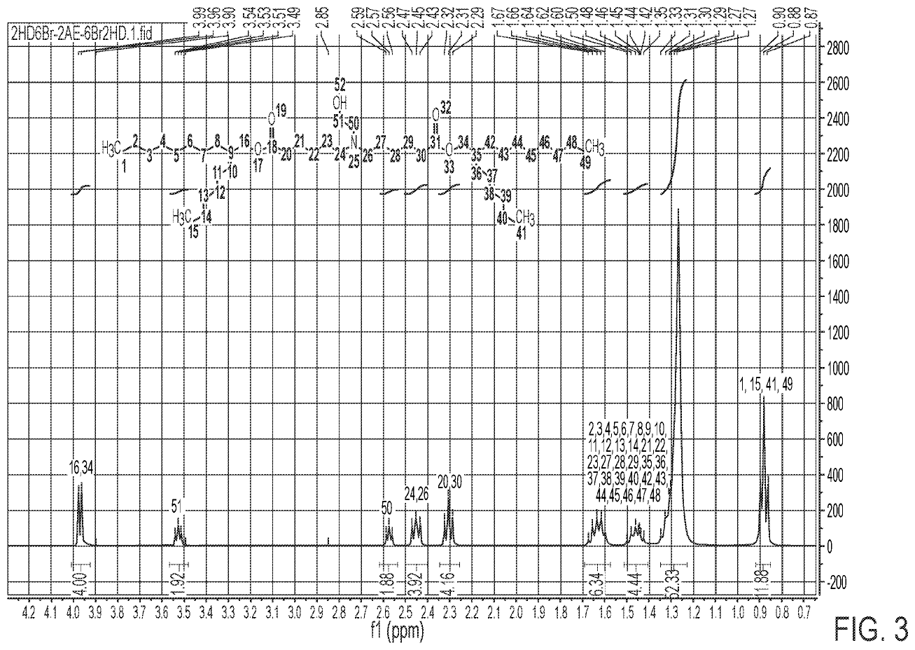

Control 1 was characterized by proton nuclear magnetic resonance on a 400 MHz spectrometer in CDCl₃, the results of which are depicted in FIG. 3. In particular, the data reported includes: chemical shift (6 ppm), multiplicity, integration, and coupling constant (Hz). ¹H NMR (400 MHz, CDCl₃) δ 3.97 (d, J=5.8 Hz, 4H), 3.53 (t, J=5.3 Hz, 2H), 2.57 (t, J=5.4 Hz, 2H), 2.45 (m, 4H), 2.31 (t, J=7.5 Hz, 4H), 1.63 (h, J=8.9, 8.2 Hz, 6H), 1.45 (m, 4H), 1.28 (m, 52H), 0.88 (t, J=6.7 Hz, 12H).

Control 1 was also characterized by electrospray ionization mass spectrometry (ESI-MS). The m/z ratio calculated for Control 1 [MH⁺] was 738.6957 for C₄₆H₉₁NO₅.

Thionyl chloride (3 equiv.) was added dropwise to a solution of Control 1 (1 equiv.) in chloroform at room temperature. The mixture was stirred for two days at room temperature and then concentrated in vacuo. The mixture was extracted using ethyl acetate and washed with brine. The organic layers were separated and dried using sodium sulfate. The organic layer was concentrated in vacuo to recover Product B, 1,1′-bis(2-hexyldecyl) 6,6′-[(2-chloroethyl)imino]bis [hexanoate].

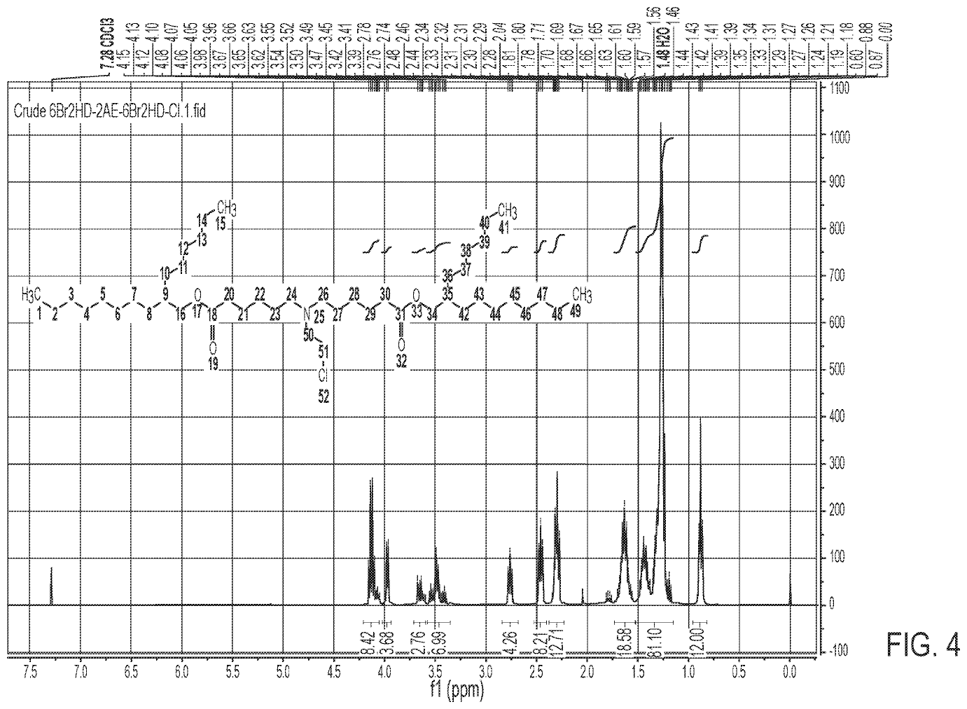

Product B was characterized by ESI-MS. The m/z ratio calculated for Product B [MH⁺] was 756.6674 for C₄₆H₉₀ClNO₄. Product B was also characterized by proton nuclear magnetic resonance on a 400 MHz spectrometer in CDCl₃, the results of which are depicted in FIG. 4. In particular, the data reported includes: chemical shift (6 ppm), multiplicity, integration, and coupling constant (Hz). ¹H NMR (400 MHz, CDCl₃) δ 4.13 (q, J=7.1 Hz, 4H), 3.97 (d, J=5.8 Hz, 2H), 3.65 (m, 1H), 3.48 (m, 3H), 2.76 (t, J=7.3 Hz 4H), 2.46 (m, 4H), 2.30 (td, J=7.5, 4.0 Hz, 6H), 1.64 (m, 9H), 1.30 (m, 38H), 0.88 (t, J=6.7 Hz, 6H).

### Example 2: Synthesis of Control 2

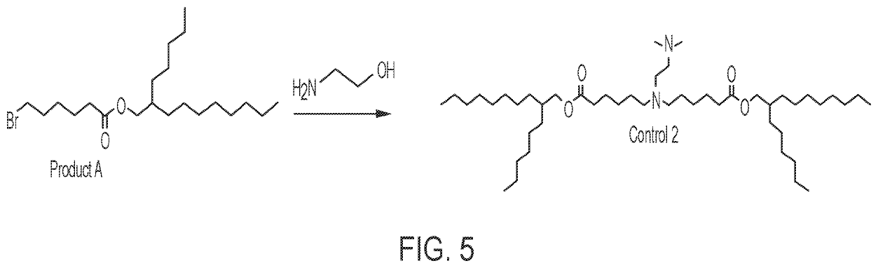

As depicted in FIG. 5, Product A (2 equiv.) was added to a solution of N,N-dimethylethylenediamine (1 equiv.) in ethanol. Potassium carbonate (2 equiv.), cesium carbonate (0.5 equiv.) and potassium iodide (1 equiv.) were added to the reaction mixture. The resulting mixture was stirred at 75° C. for two days. The mixture was then concentrated in vacuo. Ethyl acetate was added, and the mixture was extracted using saturated sodium bicarbonate and brine. The organic layers were dried using sodium sulfate and the resultant 1,1′-bis(2-hexyldecyl) 6,6′-[[2-(dimethylamino)ethyl]imino]bis[hexanoate](Control 2) was purified using column chromatography (0-20% (mixture of 10% NH₄OH in methanol) in dichloromethane).

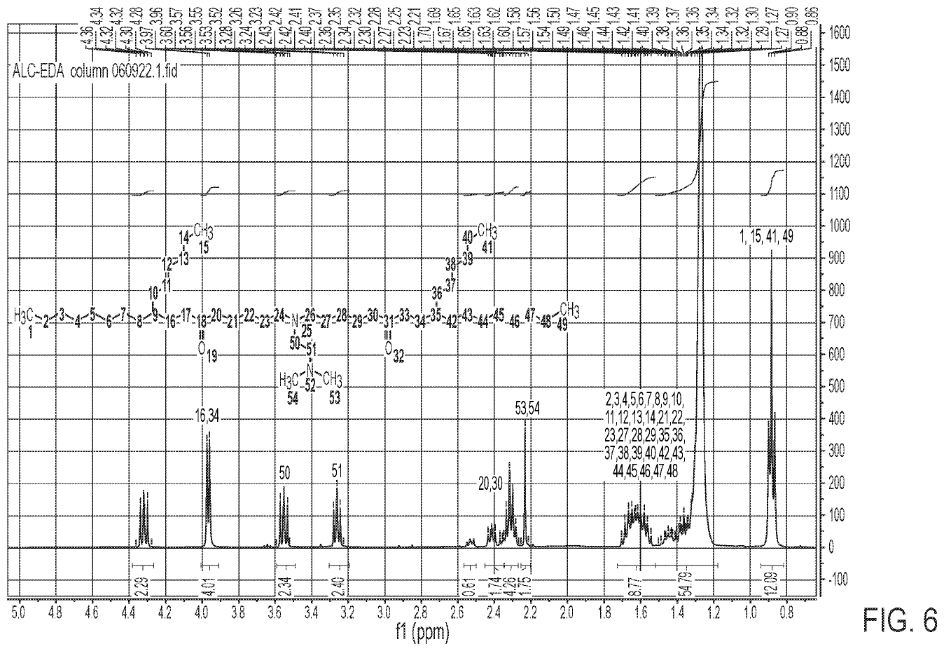

Control 2 was characterized by proton nuclear magnetic resonance on a 400 MHz spectrometer in CDCl₃, the results of which are depicted in FIG. 6. In particular, the data reported includes: chemical shift (6 ppm), multiplicity, integration, and coupling constant (Hz). ¹H NMR (400 MHz, CDCl₃) δ 4.32 (dd, J=8.9, 7.1 Hz, 2H), 3.97 (d, J=5.8 Hz, 4H), 3.55 (dd, J=8.8, 7.1 Hz, 2H), 3.26 (t, J=7.3 Hz, 2H), 2.46-2.26 (m, 6H), 2.23 (s, 2H), 1.71-1.52 (m, 8H), 1.51-1.24 (m, 52H), 0.88 (t, J=6.7 Hz, 12H).

Control 2 was also characterized and confirmed by ESI-MS. The m/z ratio calculated for Control 2 [MH⁺] was 765.7417 for C₄₈H₉₆N₂O₄.

Thionyl chloride (3 equiv.) was added dropwise to a solution of Control 1 (1 equiv.) in chloroform at room temperature. The mixture was stirred for two days at room temperature and then concentrated in vacuo. The mixture was extracted using ethyl acetate and washed with brine. The organic layers were separated and dried using sodium sulfate. The organic layer was concentrated in vacuo to recover Product B, 1,1′-bis(2-hexyldecyl) 6,6′-[(2-chloroethyl)imino]bis [hexanoate].

Product B was characterized by ESI-MS. The m/z ratio calculated for Product B [MH⁺] was 756.6674 for C₄₆H₉₀ClNO₄. Product B was also characterized by proton nuclear magnetic resonance on a 400 MHz spectrometer in CDCl₃, the results of which are depicted in FIG. 4. In particular, the data reported includes: chemical shift (8 ppm), multiplicity, integration, and coupling constant (Hz). ¹H NMR (400 MHz, CDCl₃) δ 4.13 (q, J=7.1 Hz, 4H), 3.97 (d, J=5.8 Hz, 2H), 3.65 (m, 1H), 3.48 (m, 3H), 2.76 (t, J=7.3 Hz 4H), 2.46 (m, 4H), 2.30 (td, J=7.5, 4.0 Hz, 6H), 1.64 (m, 9H), 1.30 (m, 38H), 0.88 (t, J=6.7 Hz, 6H).

### Example 3: Synthesis of Lipid 1

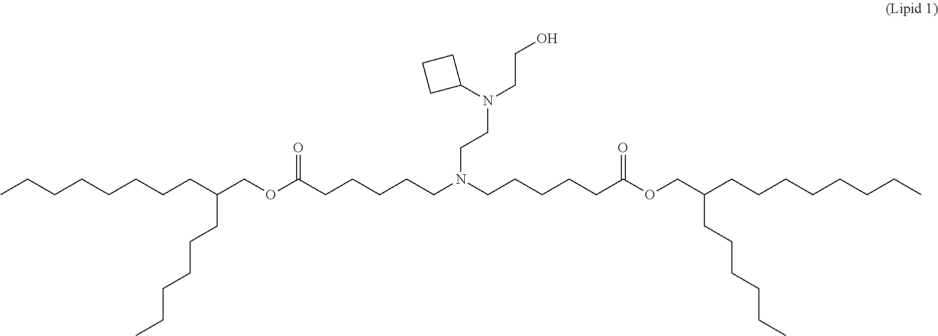 [SMILES: CCCCCCCCC(CCCCCC)COC(=O)CCCCCN(CCCCCC(=O)OCC(CCCCCC)CCCCCCCC)CCN(CCO)C1CCC1]

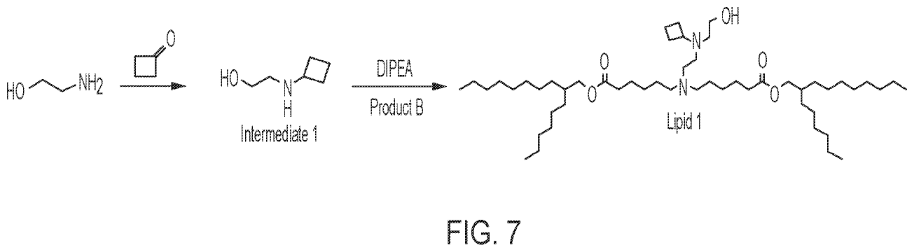

As depicted in FIG. 7, cyclobutanone (1 equiv.), titanium (IV) isopropoxide (1.3 equiv.), and 2-aminoethanol (3 equiv.) in methanol were stirred overnight at room temperature. Sodium borohydride (1.1 equiv.) was added and stirred for 6 hours. The reaction was quenched by adding water. The reaction mixture was extracted using ethyl acetate. The organic layer was separated, dried and charged into a silica column to purify and yield Intermediate 1. Intermediate 1 was characterized and confirmed using ESI-MS. The m/z ratio calculated for Intermediate 1 [MH⁺] was 116.1065 for C₆H₁₃NO.

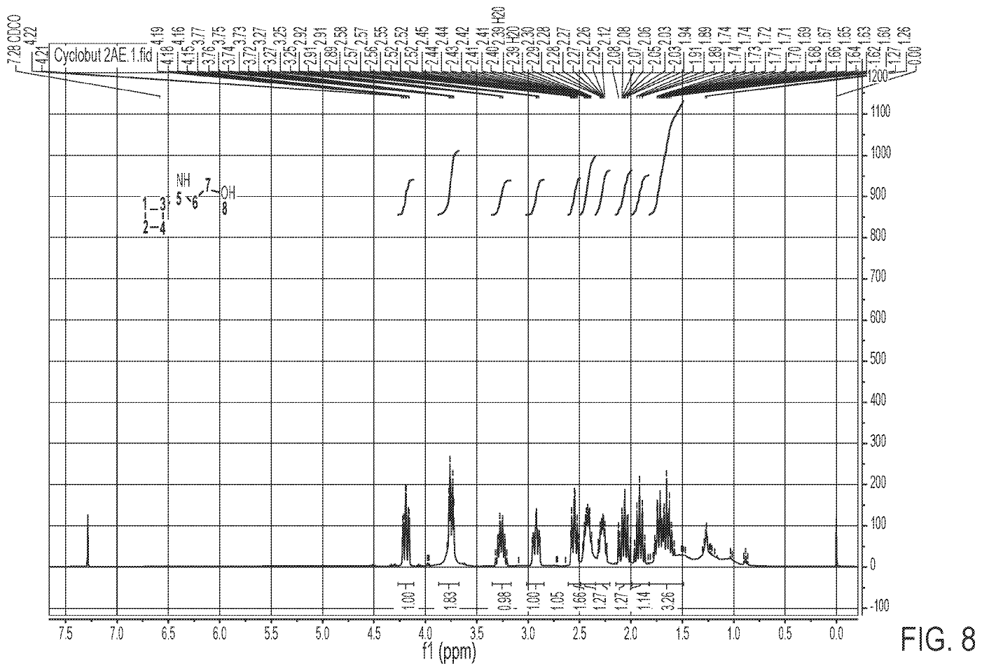

Intermediate 1 was characterized by proton nuclear magnetic resonance on a 400 MHz spectrometer in CDCl₃, the results of which are depicted in FIG. 8. In particular, the data reported includes: chemical shift (6 ppm), multiplicity, integration, and coupling constant (Hz). ¹H NMR (400 MHz, CDCl₃) δ 4.19 (td, J=11.0, 2.9 Hz, 1H), 3.74 (dt, J=11.4, 3.5 Hz, 2H), 3.35-3.16 (m, 1H), 2.92 (dddd, J=12.5, 10.5, 3.8, 2.2 Hz, 1H), 2.55 (ddt, J=12.5, 9.6, 3.0 Hz, 1H), 2.42 (ddt, J=12.7, 10.1, 6.4 Hz, 2H), 2.34-2.20 (m, 1H), 2.15-1.99 (m, 1H), 1.91 (p, J=10.2 Hz, 1H), 1.82-1.48 (m, 3H).

Product B (1 equiv.) in ethanol was added to a solution of Intermediate 1 (2.5 equiv.). N,N-diisopropylethylamine (DIPEA) (5 equiv.) and potassium iodide (1 equiv.) was added and the mixture was stirred at 75° C. for 16 hours. The reaction mixture was concentrated in vacuo. Ethyl acetate was added to the mixture. The mixture was extracted using saturated sodium thiosulfate, sodium bicarbonate and brine. The organic layers were separated and dried, after which they were charged into a column for separation using column chromatography (0-20% (mixture of 10% NH₄OH in methanol) in dichloromethane). Resultant Lipid 1 was characterized and confirmed using ESI-MS. The m/z ratio calculated for Lipid 1 [MH⁺] was 835.7862 for C₅₂H₁₀₂N₂O₅.

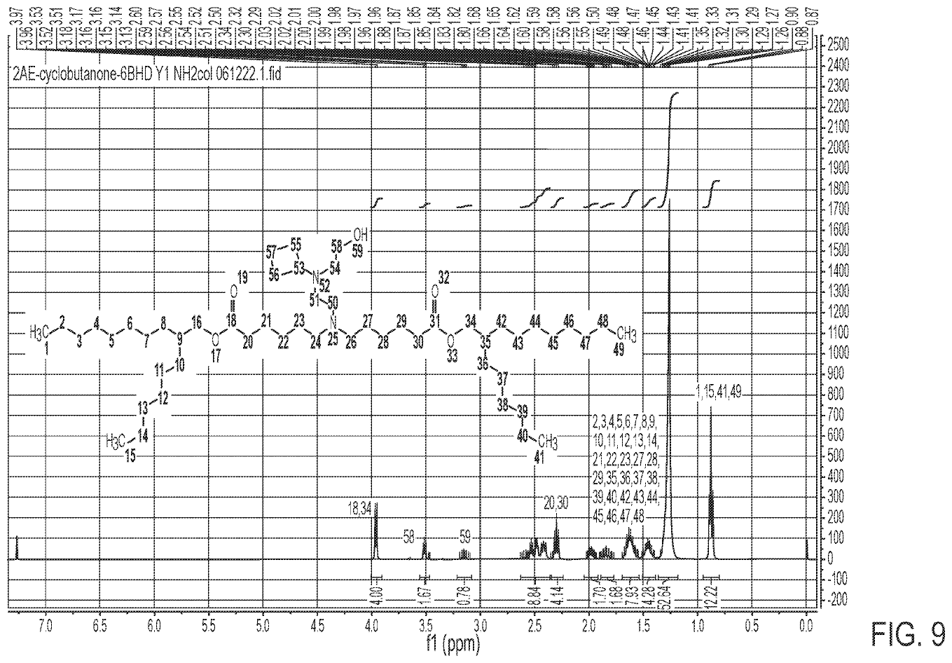

Lipid 1 (Control 3) was also characterized by proton nuclear magnetic resonance on a 400 MHz spectrometer in CDCl₃, the results of which are depicted in FIG. 9. In particular, the data reported includes: chemical shift (6 ppm), multiplicity, integration, and coupling constant (Hz). ¹H NMR (400 MHz, CDCl₃) δ 3.96 (d, J=5.8 Hz, 4H), 3.52 (t, J=5.1 Hz, 2H), 3.16 (tt, J=8.9, 6.8 Hz, 1H), 2.63-2.36 (m, 9H), 2.30 (t, J=7.5 Hz, 4H), 1.99 (ddt, J=9.6, 7.1, 4.0 Hz, 2H), 1.90-1.78 (m, 2H), 1.62 (tt, J=18.4, 9.2 Hz, 8H), 1.52-1.39 (m, 4H), 1.37-1.19 (m, 53H), 0.88 (t, J=6.7 Hz, 12H).

### Example 4: Synthesis of Lipid 2

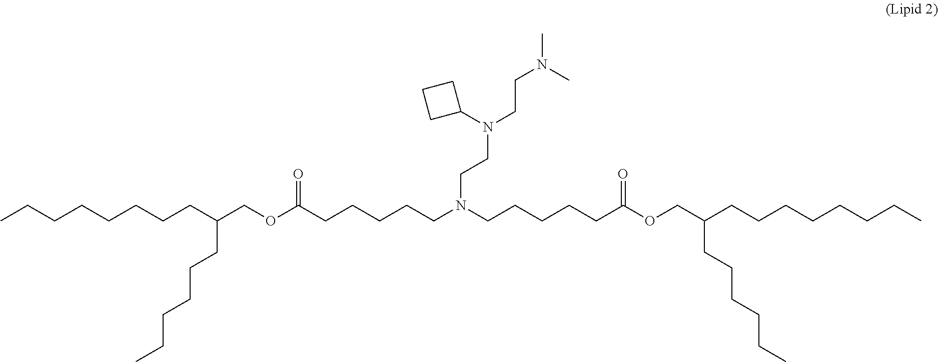 [SMILES: CCCCCCCCC(CCCCCC)COC(=O)CCCCCN(CCCCCC(=O)OCC(CCCCCC)CCCCCCCC)CCN(CCN(C)C)C1CCC1]

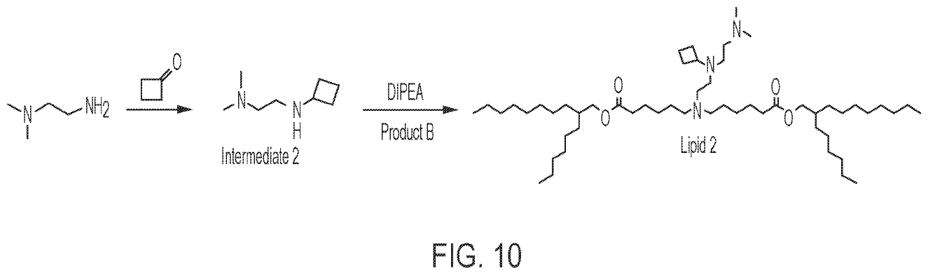

As depicted in FIG. 10, cyclobutanone (1 equiv.), titanium (IV) isopropoxide (1.3 equiv.), and N,N-dimethylethylenediamine (1.5 equiv.) in methanol were stirred overnight at room temperature. Sodium borohydride (1.1 equiv.) was added and stirred for 6 hours. The reaction was quenched by adding water. The reaction mixture was extracted using ethyl acetate. The organic layer was separated, dried and charged into a silica column to purify and yield Intermediate 2. Intermediate 2 was characterized and confirmed using ESI-MS. The m/z ratio calculated for Intermediate 2 [MH⁺] was 143.1537 for C₈H₁₈N₂.

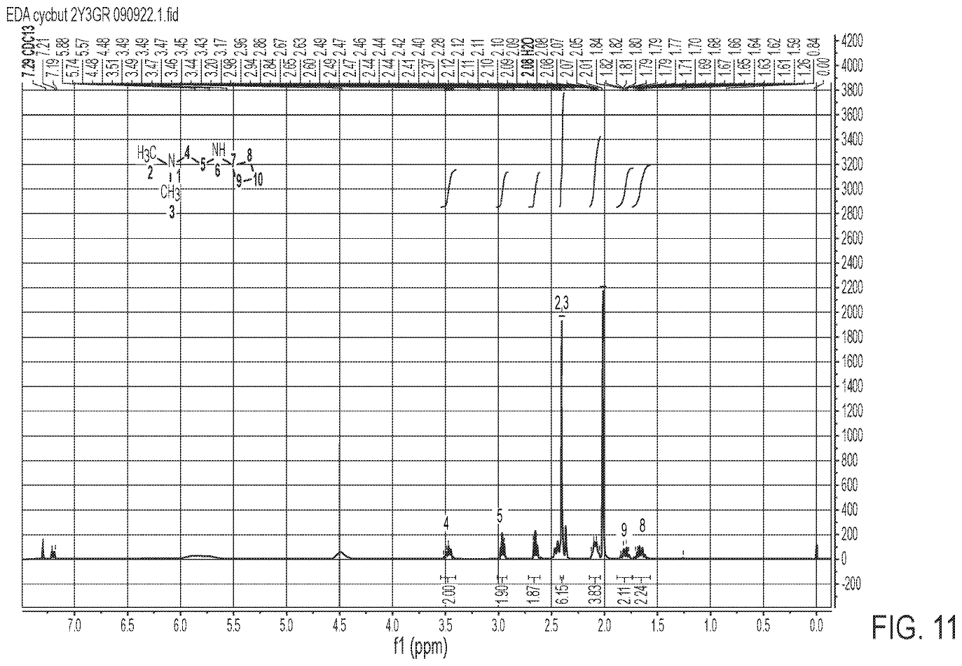

Intermediate 2 was characterized by proton nuclear magnetic resonance on a 400 MHz spectrometer in CDCl₃, the results of which are depicted in FIG. 11. In particular, the data reported includes: chemical shift (δ ppm), multiplicity, integration, and coupling constant (Hz). ¹H NMR (400 MHz, CDCl₃) δ ¹H NMR (400 MHz, CDCl3) δ 3.54-3.40 (m, 2H), 2.96 (t, J=6.9 Hz, 2H), 2.65 (t, J=6.9 Hz, 2H), 2.40 (s, 6H), 2.14-2.04 (m, 4H), 1.88-1.74 (m, 2H), 1.66 (qt, J=10.8, 7.9 Hz, 2H).

Product B (1 equiv.) in ethanol was added to a solution of Intermediate 2 (2.5 equiv.). N,N-diisopropylethylamine (DIPEA) (5 equiv.) and potassium iodide (1 equiv.) were added and the mixture was stirred at 75° C. for sixteen hours. The reaction mixture was concentrated in vacuo. Ethyl acetate was added to the mixture. The mixture was extracted using saturated sodium thiosulfate, sodium bicarbonate, and brine. The organic layers were separated and dried, after which they were charged into a column for separation using column chromatography (0-20% (mixture of 10% NH₄OH in methanol) in dichloromethane). Resultant Lipid 2 was characterized and confirmed using ESI-MS. The m/z ratio calculated for Lipid 2 [MH⁺] was 862.8306 for C₅₄H₁₀₇N₃O₄.

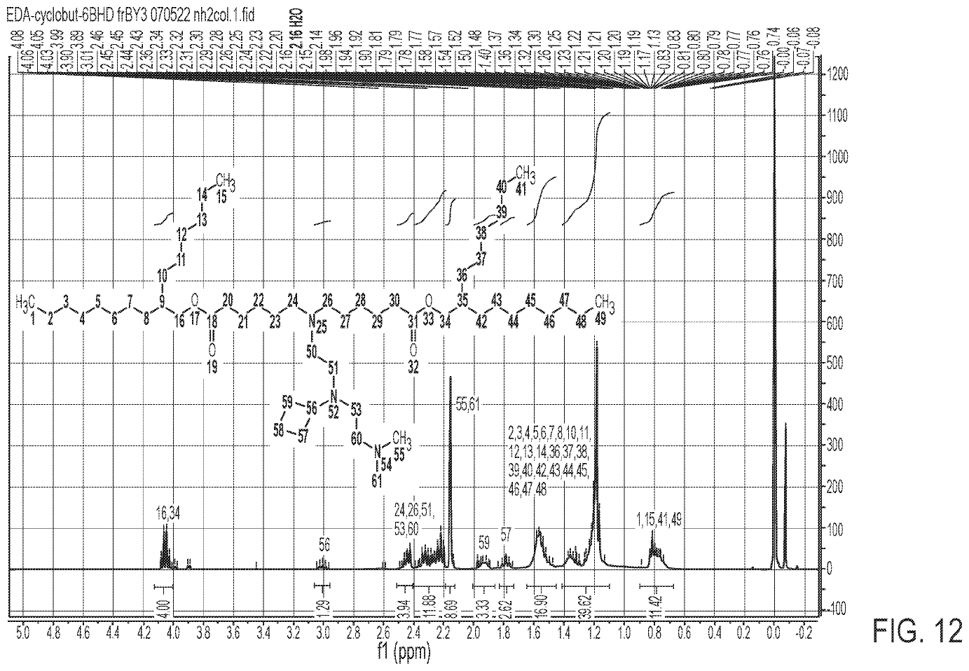

Lipid 2 was also characterized by proton nuclear magnetic resonance on a 400 MHz spectrometer in CDCl₃, the results of which are depicted in FIG. 12. In particular, the data reported includes: chemical shift (6 ppm), multiplicity, integration, and coupling constant (Hz). ¹H NMR (400 MHz, CDCl₃) δ 4.06 (q, J=7.2 Hz, 4H), 3.01 (p, J=7.8 Hz, 1H), 2.51-2.40 (m, 4H), 2.40-2.19 (m, 12H), 2.19-2.13 (m, 9H), 2.00-1.86 (m, 3H), 1.83-1.73 (m, 3H), 1.65-1.45 (m, 17H), 1.41-1.09 (m, 40H), 0.89-0.67 (m, 12H).

### Example 5: Synthesis of Lipid 3

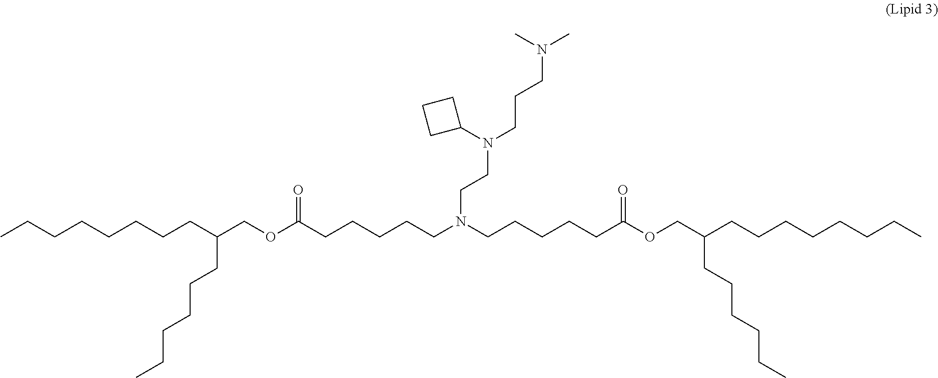 [SMILES: CCCCCCCCC(CCCCCC)COC(=O)CCCCCN(CCCCCC(=O)OCC(CCCCCC)CCCCCCCC)CCN(CCCN(C)C)C1CCC1]

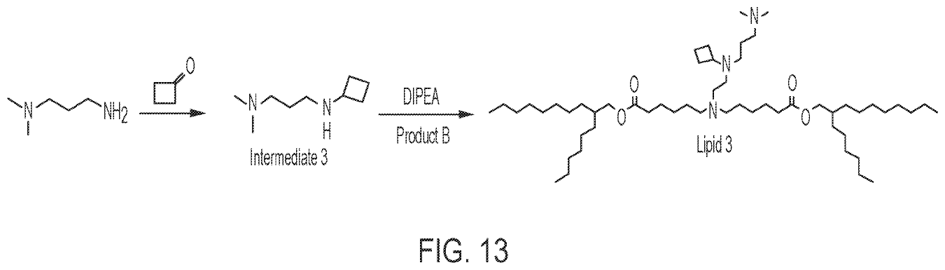

As depicted in FIG. 13, cyclobutanone (1 equiv.), titanium (IV) isopropoxide (1.3 equiv.), and 3-(dimethylamino)-1-propylamine (2 equiv.) in methanol in methanol were stirred overnight at room temperature. Sodium borohydride (1.1 equiv.) was added and stirred for 6 hours. The reaction was quenched by adding water. The reaction mixture was extracted using ethyl acetate. The organic layer was separated, dried and charged into a silica column to purify and yield Intermediate 3. Intermediate 3 was characterized and confirmed using ESI-MS. The m/z ratio calculated for Intermediate 3 [MH⁺] was 157.1696 for C₉H₂₀N₂.

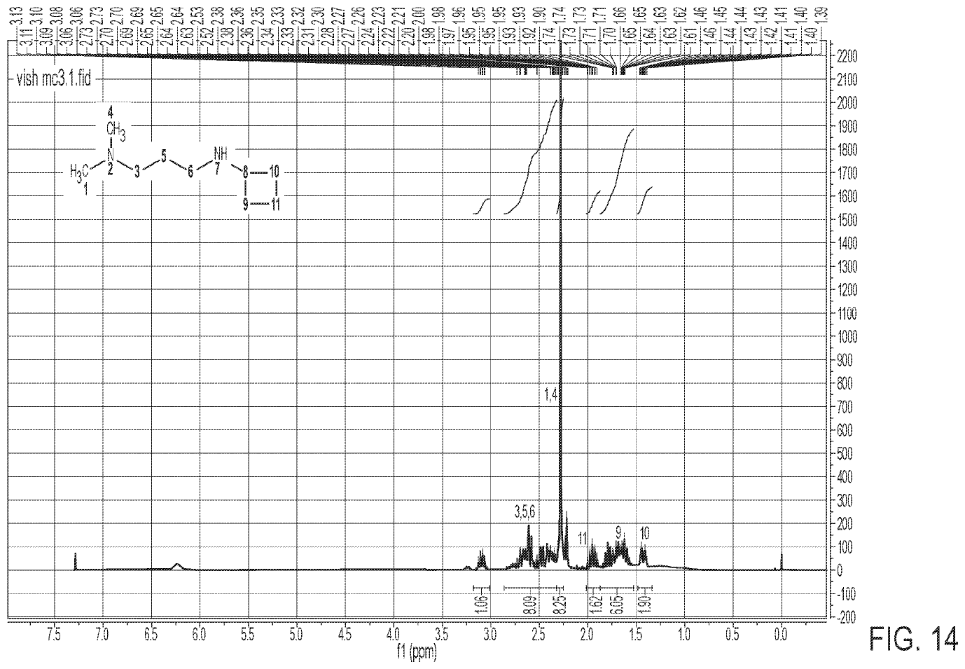

Intermediate 3 was characterized by proton nuclear magnetic resonance on a 400 MHz spectrometer in CDCl₃, the results of which are depicted in FIG. 14. In particular, the data reported includes: chemical shift (6 ppm), multiplicity, integration, and coupling constant (Hz). ¹H NMR (400 MHz, CDCl₃) δ 3.09 (dt, J=9.8, 7.4 Hz, 1H), 2.86-2.32 (m, 8H), 2.28 (s, 8H), 1.95 (tdd, J=11.4, 10.0, 8.9 Hz, 2H), 1.87-1.53 (m, 6H), 1.48-1.34 (m, 2H).

Product B (1 equiv.) in ethanol was added to a solution of Intermediate 3 (2.5 equiv.). N,N-diisopropylethylamine (DIPEA) (5 equiv.) and potassium iodide (1 equiv.) were added and the mixture was stirred at 75° C. for sixteen hours. The reaction mixture was concentrated in vacuo. Ethyl acetate was added to the mixture. The mixture was extracted using saturated sodium thiosulfate, sodium bicarbonate, and brine. The organic layers were separated and dried, after which they were charged into a column for separation using column chromatography (0-20% (mixture of 10% NH₄OH in methanol) in dichloromethane). Resultant Lipid 3 was characterized and confirmed using ESI-MS. The m/z ratio calculated for Lipid 3 [MH⁺] was +) 876.8495 for C₅₅H₁₀₉N₃O₄.

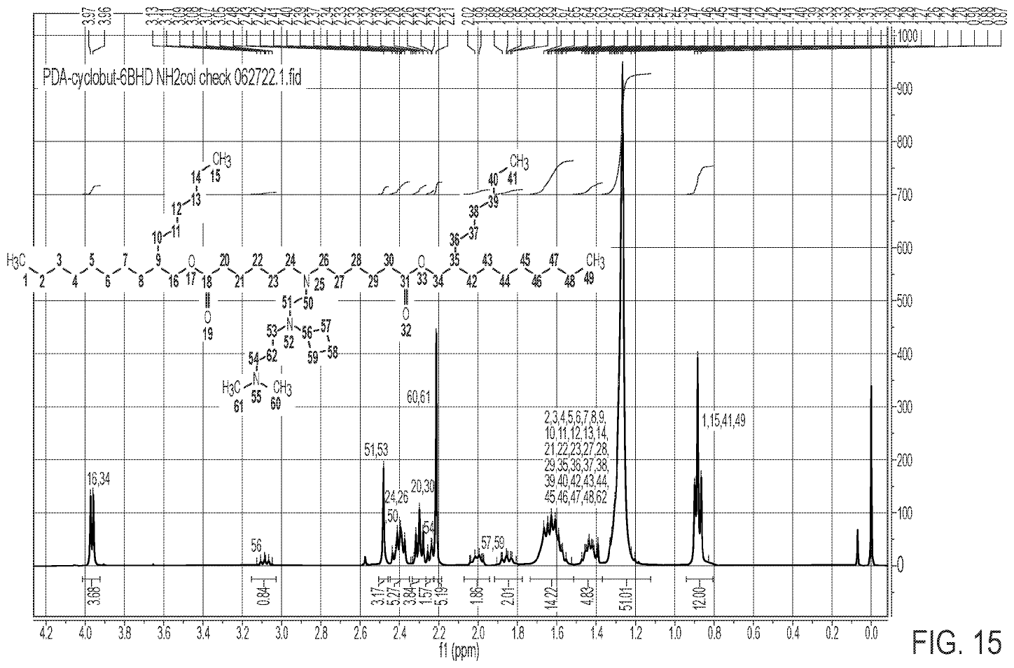

Lipid 3 was also characterized by proton nuclear magnetic resonance on a 400 MHz spectrometer in CDCl₃, the results of which are depicted in FIG. 15. In particular, the data reported includes: chemical shift (6 ppm), multiplicity, integration, and coupling constant (Hz). ¹H NMR (400 MHz, CDCl₃) δ 3.96 (d, J=5.8 Hz, 4H), 3.15-3.03 (m, 1H), 2.48 (s, 3H), 2.40 (td, J=9.2, 6.3 Hz, 5H), 2.30 (t, J=7.5 Hz, 6H), 2.26-2.22 (m, 2H), 2.21 (s, 5H), 2.07-1.94 (m, 2H), 1.85 (qd, J=10.5, 8.2 Hz, 2H), 1.62 (td, J=15.0, 7.5 Hz, 14H), 1.51-1.37 (m, 5H), 1.37-1.12 (m, 51H), 0.88 (t, J=6.7 Hz, 12H).

### Example 6: Preparation of Lipid Nanoparticles

Different lipid nanoparticles (LNPs) were formulated by incorporating the different ionizable lipids and controls from Examples 1-5.1. SM102 LNPs were prepared as a control. The lipids were formulated with the ionizable lipids, DOPE, cholesterol, and DMG-PEG.

The ionizable lipids, DOPE, cholesterol and DMG-PEG were dissolved in ethanol at a concentration of 2 mg/ml and were mixed at a molar ratio of 40:10:48:2 to create a lipid mixture.

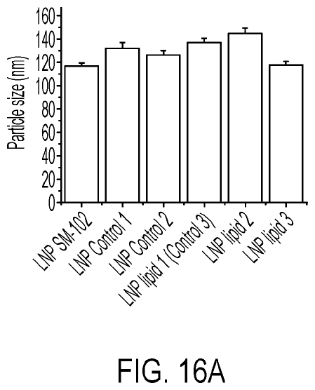

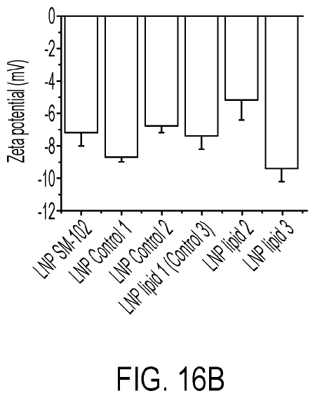

A molar ratio of 8:1 was used between the amine group of the ionizable lipid and the phosphate group of the mRNA. mRNA diluted in 5 mM citrate buffer (pH 5.0) was mixed with the lipid mixture at a 3:1 volume ratio. After incubating the sample for 30 minutes, the solution was concentrated using an Amicon® filter (MilliporeSigma, Burlington, MA, MWCO: 30,000 Da) to remove the ethanol. The encapsulation efficiency of mRNA was determined using QuantiFluor® RNA system (Promega). Particle size and surface charge of the LNPs were determined using Nanobrook Omni, the results of which are demonstrated in FIGS. 16A-16B.

The LNPs were characterized for particle size and surface charge using dynamic light scattering and zeta potential measurements (FIGS. 16A-16B). LNPs showed a range of particle size of from ˜110 to ˜150 nm (FIG. 16A). The surface charge of the LNPs ranged from −5 mV to −10 mV (FIG. 16B). Since these LNPs contained ionizable lipids, their surface charge was close to neutral when they were measured at physiological pH of 7.4.

### Example 7: In Vitro Cellular Uptake and Transfection Efficiencies

Flow cytometry was used to determine in vitro cellular uptake and transfection efficacy in human Jurkat T cells. The cells were seeded in a 96-well plate at a density of 40,000 cells/well. Different formulations containing 100 ng of mRNA were added to each well and were incubated for 44 hours at 37° C. The cells were washed and centrifuged at 300×g. The supernatant was discarded. After the cells were diluted in PBS, flow cytometry analysis was performed to quantify the amounts of cellular uptake and transfection efficiency.

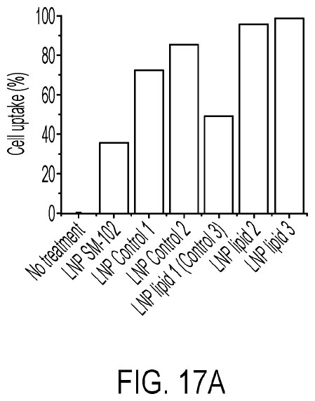

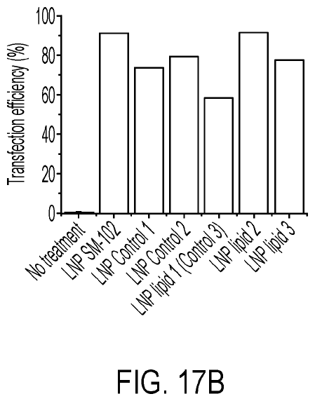

LNPs with different compositions were tested against Jurkat cells for both cellular uptake and mRNA transfection efficiencies, as depicted in FIGS. 17A and 17B respectively. Human Jurkat cells are representative T lymphocytes which are known to be difficult to transfect. The LNPs were tested for cellular uptake using Cy5-labeled eGFP mRNA. Cy5 fluorescent dye was used to determine the cellular uptake while eGFP fluorescence was used to determine the transfection efficiency of the mRNA encapsulated in the different LNP formulations. After 44-hour incubation, fluorescence was measured using a flow cytometer. In Jurkat cells, the cellular uptake was ˜36% for the positive control, SM-102 while the transfection efficiency was ˜91%. Most LNPs showed good cell uptake results. Control 2 and Lipid 1 (Control 3) showed a transfection efficiency of 80 and 59% respectively. Lipid 2 and Lipid 3 showed a transfection efficiency of 92 and 78% respectively.

Notably, as shown in FIGS. 17A, Lipid 2 and Lipid 3 outperformed the controls in cellular uptake efficiency and transfection efficiency by a wide margin in the Jurkat cells.

### FURTHER EXAMPLES

A first item of the present disclosure, either alone or in combination with any other item herein, concerns a composition comprising at least one ionizable lipid according to General Formula (I),

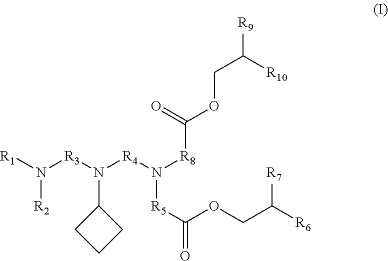

or a pharmaceutically-acceptable salt thereof, in which: R₁ is independently selected from the group consisting of C₁ to C₂ alkyl groups; R₂ is independently selected from the group consisting of C₁ to C₂ alkyl groups; R₃ is independently selected from the group consisting of C₂ to C₄ alkyl groups; R₄ is independently selected from the group consisting of C₂ to C₄ alkyl groups; R₅ is independently selected from the group consisting of C₂ to C₈ alkyl groups; R₆ is independently selected from the group consisting of C₁ to C₁₂ alkyl groups; R₇ is independently selected from the group consisting of C₁ to C₁₂ alkyl groups; R₈ is independently selected from the group consisting of C₂ to C₈ alkyl groups; R₉ is independently selected from the group consisting of C₁ to C₁₂ alkyl groups; and R₁₀ is independently selected from the group consisting of C₁ to C₁₂ alkyl groups.

A second item of the present disclosure, either alone or in combination with any other item herein, concerns a wherein the ionizable lipid is selected from the group consisting of:

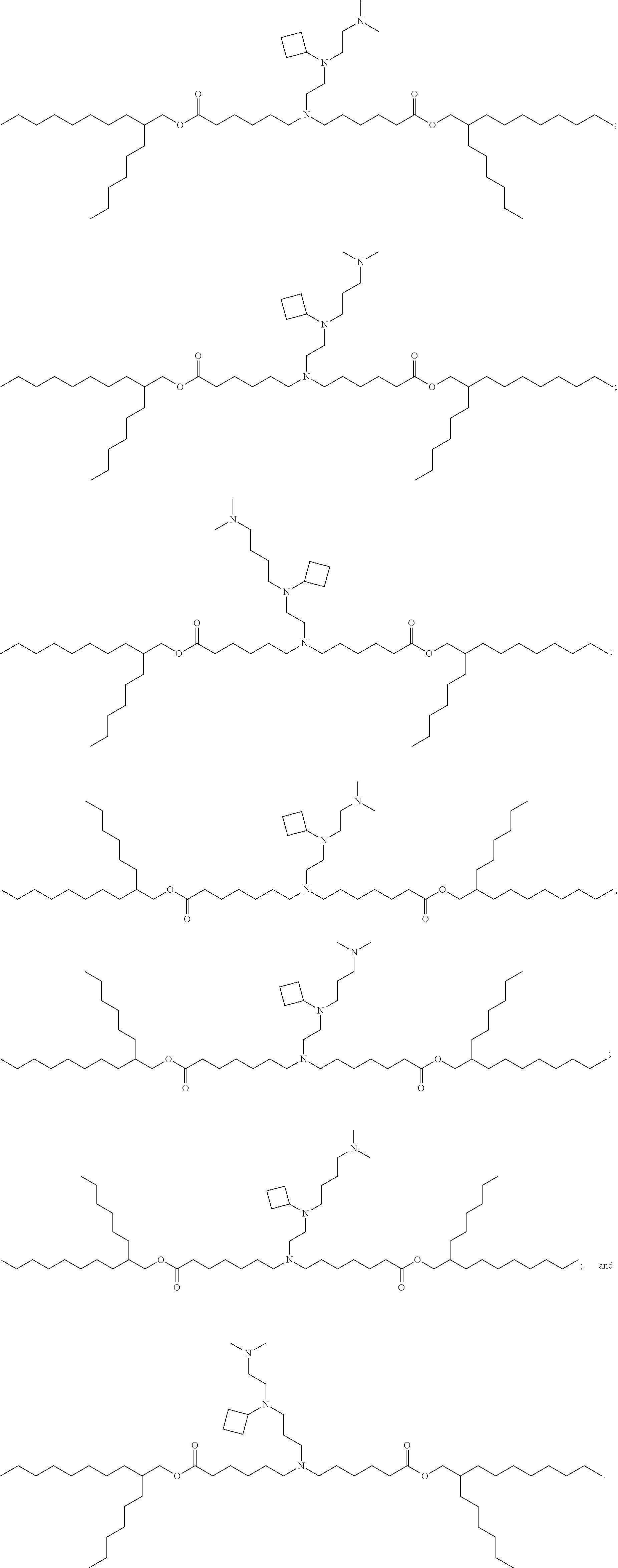

A third item of the present disclosure, either alone or in combination with any other item herein, concerns a composition comprising at least one ionizable lipid according to General Formula (II),

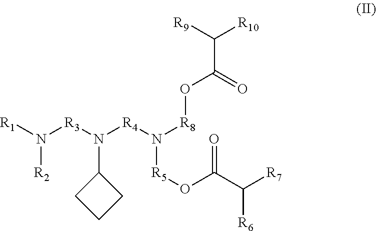

or a pharmaceutically-acceptable salt thereof, in which: R₁ is independently selected from the group consisting of C₁ to C₂ alkyl groups; R₂ is independently selected from the group consisting of C₁ to C₂ alkyl groups; R₃ is independently selected from the group consisting of C₂ to C₄ alkyl groups; R₄ is independently selected from the group consisting of C₂ to C₄ alkyl groups; R₅ is independently selected from the group consisting of C₂ to C₈ alkyl groups; R₆ is independently selected from the group consisting of —H, and C₁ to C₁₂ alkyl groups; R₇ is independently selected from the group consisting of C₁ to C₁₂ alkyl groups; R₈ is independently selected from the group consisting of C₂ to C₈ alkyl groups; R₉ is independently selected from the group consisting of —H and C₁ to C₁₂ alkyl groups; and R₁₀ is independently selected from the group consisting of C₁ to C₁₂ alkyl groups.

A fourth item of the present disclosure, either alone or in combination with any other item herein, concerns a composition wherein the ionizable lipid is selected from the group consisting of:

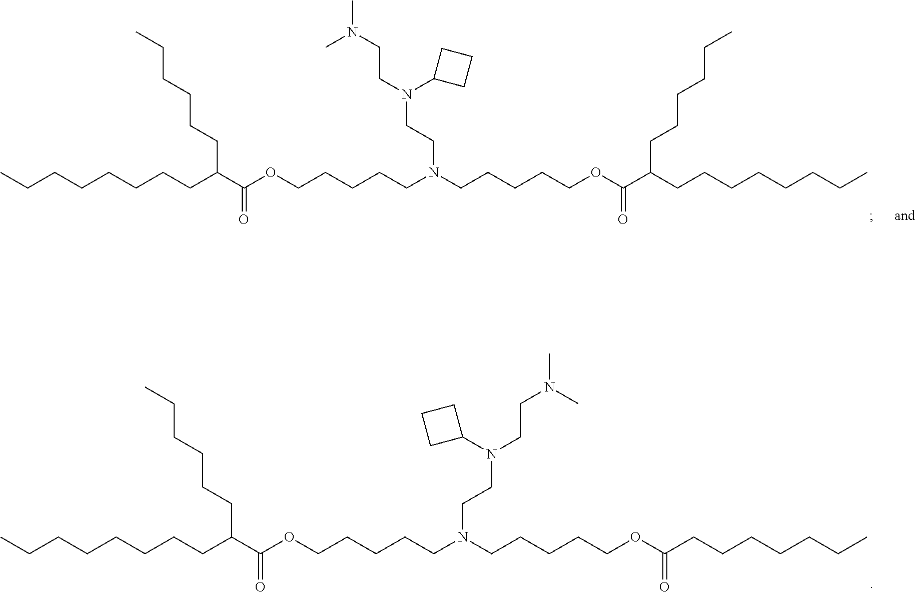

A fifth item of the present disclosure, either alone or in combination with any other item herein, concerns a composition comprising at least one ionizable lipid according to General Formula (III),

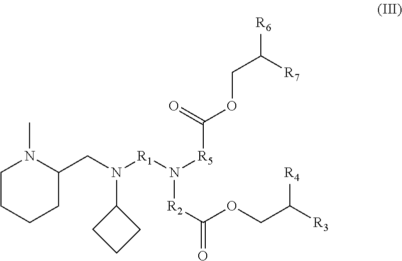

or a pharmaceutically-acceptable salt thereof, in which: R₁ is independently selected from the group consisting of C₂ to C₄ alkyl groups; R₂ is independently selected from the group consisting of C₂ to C₈ alkyl groups; R₃ is independently selected from the group consisting of C₁ to C₁₂ alkyl groups; R₄ is independently selected from the group consisting of C₁ to C₁₂ alkyl groups; R₅ is independently selected from the group consisting of C₂ to C₈ alkyl groups; R₆ is independently selected from the group consisting of C₁ to C₁₂ alkyl groups; and R₇ is independently selected from the group consisting of C₁ to C₁₂ alkyl groups.

A sixth item of the present disclosure, either alone or in combination with any other item herein, concerns a composition of, wherein the ionizable lipid is:

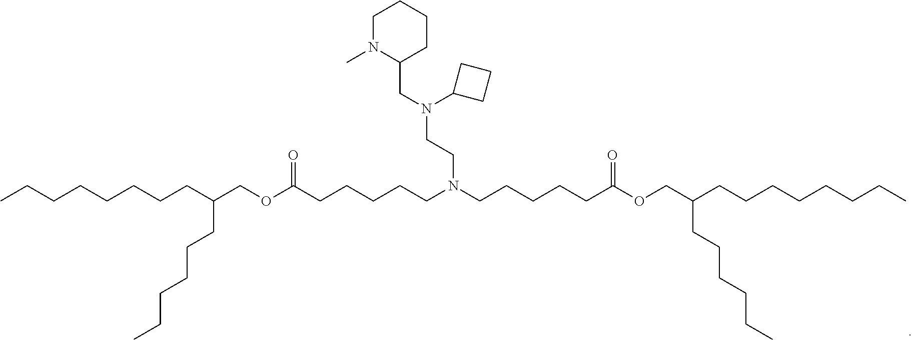 [SMILES: CCCCCCCCC(CCCCCC)COC(=O)CCCCCN(CCCCCC(=O)OCC(CCCCCC)CCCCCCCC)CCN(CC1CCCCN1C)C1CCC1]

A seventh item of the present disclosure, either alone or in combination with any other item herein, concerns a composition comprising at least one ionizable lipid according to General Formula (IV),

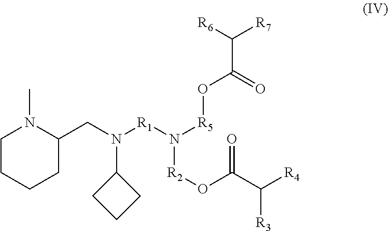

or a pharmaceutically-acceptable salt thereof, in which: R₁ is independently selected from the group consisting of C₂ to C₄ alkyl groups; R₂ is independently selected from the group consisting of C₂ to C₈ alkyl groups; R₃ is independently selected from the group consisting of C₁ to C₁₂ alkyl groups; R₄ is independently selected from the group consisting of —H and C₁ to C₁₂ alkyl groups; R₅ is independently selected from the group consisting of C₂ to C₈ alkyl groups; R₆ is independently selected from the group consisting of —H, and C₁ to C₁₂ alkyl groups; and R₇ is independently selected from the group consisting of C₁ to C₁₂ alkyl groups.

An eighth item of the present disclosure, either alone or in combination with any other item herein, concerns a composition comprising an ionizable lipid and further comprising: a helper lipid; a structural lipid or sterol; and a polymer-conjugated lipid, wherein the composition forms lipid nanoparticles.

A ninth item of the present disclosure, either alone or in combination with any other item herein, concerns a composition wherein the ionizable lipid is selected from the lipids represented in Table 1.

A tenth item of the present disclosure, either alone or in combination with any other item herein, concerns a composition wherein the helper lipid is selected from the group consisting of 1, 2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1, 2-dioleoyl-sn-glycero-3-phospho-rac-(1-glycerol) sodium salt (DOPG), sphingomyelin and combinations thereof.

An eleventh item of the present disclosure, either alone or in combination with any other item herein, concerns a composition wherein the helper lipid is DSPC.

A twelfth item of the present disclosure, either alone or in combination with any other item herein, concerns a composition, wherein the helper lipid is DOPE.

A thirteenth item of the present disclosure, either alone or in combination with any other item herein, concerns a composition of claim 10, wherein the helper lipid is a combination of DSPC and DOPE.

A fourteenth item of the present disclosure, either alone or in combination with any other item herein, concerns a composition wherein the structural lipid or sterol is cholesterol or a derivative thereof.

A fifteenth item of the present disclosure, either alone or in combination with any other item herein, concerns a composition wherein the polymer-conjugated lipid is selected from the group consisting of 1-(monomethoxy-polyethyleneglycol)-2, 3-dimyristoylglycerol (PEG-DMG); pegylated phosphatidylethanoloamine (PEG-PE); 4-O-(2′,3′-di (tetradecanoyloxy) propyl-1-O-(ω-methoxy (polyethoxy) ethyl) butanedioate (PEG-S-DMG); ω-methoxy (polyethoxy) ethyl-N-(2,3-di (tetradecanoxy) propyl) carbamate; and 2,3-di(tetradecanoxy) propyl-N-(ω-methoxy (polyethoxy) ethyl) carbamate.

A sixteenth item of the present disclosure, either alone or in combination with any other item herein, concerns a composition wherein the ionizable lipid comprises from about 40-60 molar percent, the helper lipid comprises from about 10-20 molar percent, the sterol comprises from about 30-50 molar percent; and the conjugate lipid comprises from about 1-5 molar percent.

A seventeenth item of the present disclosure, either alone or in combination with any other item herein, concerns a composition wherein the lipid nanoparticle at least partially encapsulates a nucleic acid.

An eighteenth item of the present disclosure, either alone or in combination with any other item herein, concerns a composition wherein the nucleic acid is mRNA.

A nineteenth item of the present disclosure, either alone or in combination with any other item herein, concerns a composition further comprising a pharmaceutically acceptable excipient.

A twentieth item of the present disclosure, either alone or in combination with any other item herein, concerns a composition wherein the composition is formulated for administration by injection or infusion.

A twenty first item of the present disclosure, either alone or in combination with any other item herein, concerns use of the composition of any of the compositions as a as a vaccination.

A twenty-second item of the present disclosure, either alone or in combination with any other item herein, concerns use of a composition wherein the use comprises administration of the composition to a subject in need thereof.

A twenty-third item of the present disclosure, either alone or in combination with any other item herein, concerns use of a composition, wherein the administration is parenteral administration.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm”.

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A composition comprising at least one ionizable lipid according to General Formula (I), 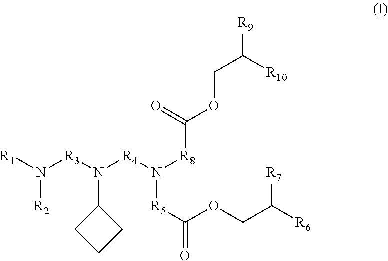
or a pharmaceutically-acceptable salt thereof, in which:
R₁ is independently selected from the group consisting of C₁ to C₂ alkyl groups;
R₂ is independently selected from the group consisting of C₁ to C₂ alkyl groups;
R₃ is independently selected from the group consisting of C₂ to C₄ alkyl groups;
R₄ is independently selected from the group consisting of C₂ to C₄ alkyl groups;
R₅ is independently selected from the group consisting of C₂ to C₈ alkyl groups;
R₆ is independently selected from the group consisting of C₁ to C₁₂ alkyl groups;
R₇ is independently selected from the group consisting of C₁ to C₁₂ alkyl groups;
R₈ is independently selected from the group consisting of C₂ to C₈ alkyl groups;
R₉ is independently selected from the group consisting of C₁ to C₁₂ alkyl groups; and
R₁₀ is independently selected from the group consisting of C₁ to C₁₂ alkyl groups.

2. The composition of claim 1, wherein the ionizable lipid is selected from the group consisting of:

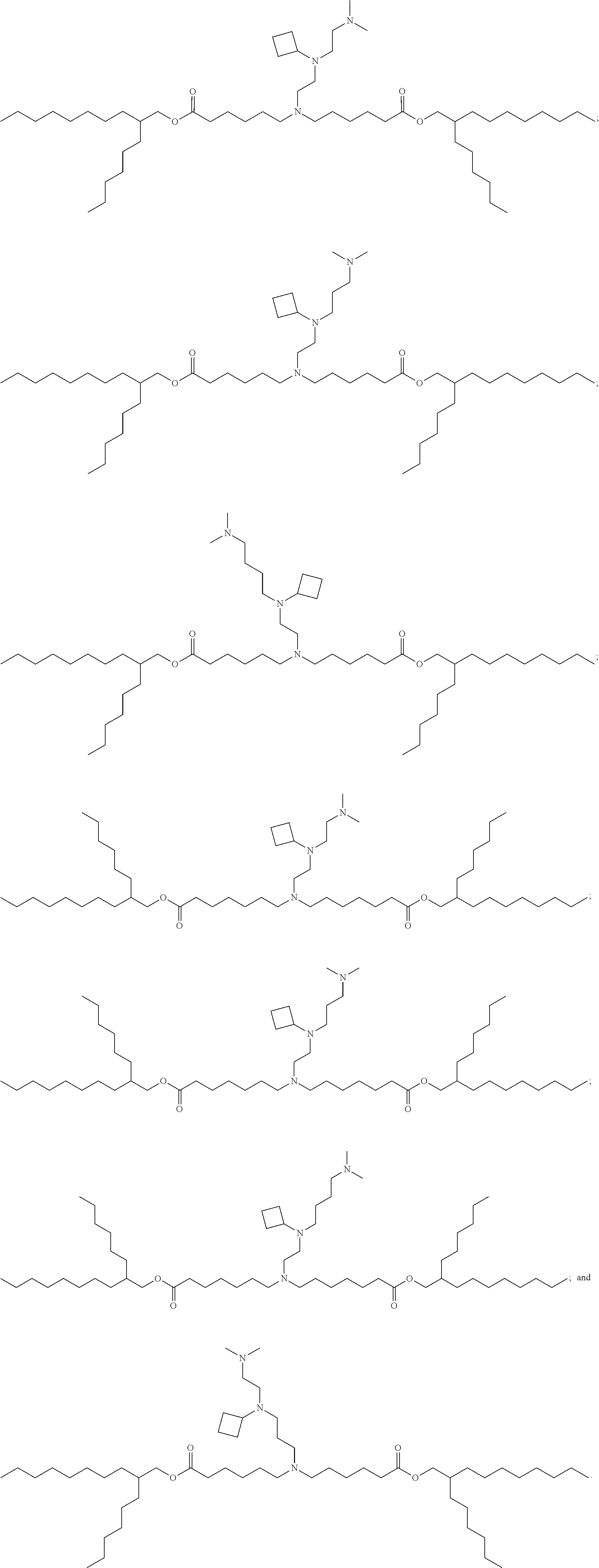

3. A composition comprising at least one ionizable lipid according to General Formula (II), 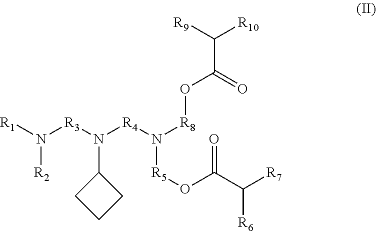
or a pharmaceutically-acceptable salt thereof, in which:
R₁ is independently selected from the group consisting of C₁ to C₂ alkyl groups;
R₂ is independently selected from the group consisting of C₁ to C₂ alkyl groups;
R₃ is independently selected from the group consisting of C₂ to C₄ alkyl groups;
R₄ is independently selected from the group consisting of C₂ to C₄ alkyl groups;
R₅ is independently selected from the group consisting of C₂ to C₈ alkyl groups;
R₆ is independently selected from the group consisting of —H, and C₁ to C₁₂ alkyl groups;
R₇ is independently selected from the group consisting of C₁ to C₁₂ alkyl groups;
R₈ is independently selected from the group consisting of C₂ to C₈ alkyl groups;
R₉ is independently selected from the group consisting of —H and C₁ to C₁₂ alkyl groups; and
R₁₀ is independently selected from the group consisting of C₁ to C₁₂ alkyl groups.

4. The composition of claim 3, wherein the ionizable lipid is selected from the group consisting of:

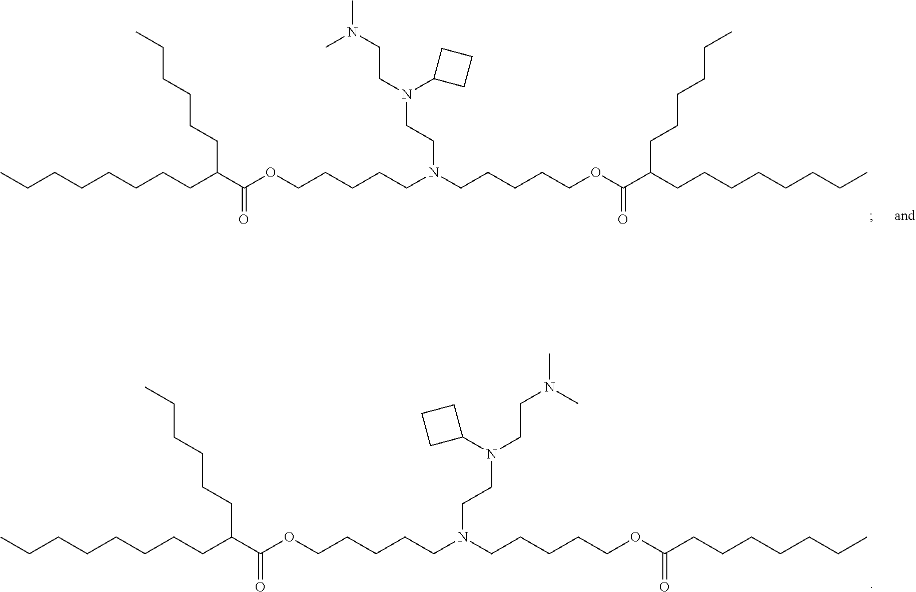

5. A composition comprising at least one ionizable lipid according to General Formula (III), 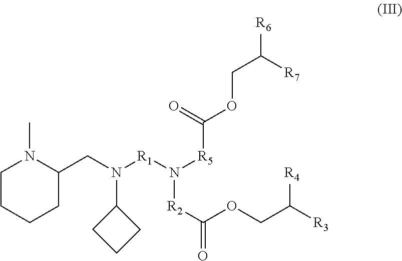
or a pharmaceutically-acceptable salt thereof, in which:
R₁ is independently selected from the group consisting of C₂ to C₄ alkyl groups;
R₂ is independently selected from the group consisting of C₂ to C₈ alkyl groups;
R₃ is independently selected from the group consisting of C₁ to C₁₂ alkyl groups;
R₄ is independently selected from the group consisting of C₁ to C₁₂ alkyl groups;
R₅ is independently selected from the group consisting of C₂ to C₈ alkyl groups;
R₆ is independently selected from the group consisting of C₁ to C₁₂ alkyl groups; and
R₇ is independently selected from the group consisting of C₁ to C₁₂ alkyl groups.

6. The composition of claim 5, wherein the ionizable lipid is:

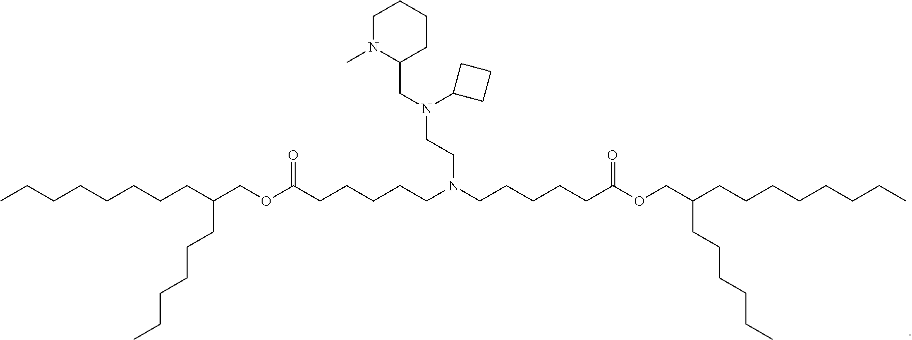 [SMILES: CCCCCCCCC(CCCCCC)COC(=O)CCCCCN(CCCCCC(=O)OCC(CCCCCC)CCCCCCCC)CCN(CC1CCCCN1C)C1CCC1]

7. A composition comprising at least one ionizable lipid according to General Formula (IV), 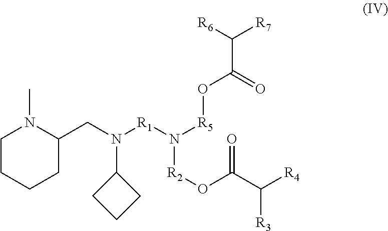
or a pharmaceutically-acceptable salt thereof, in which:
R₁ is independently selected from the group consisting of C₂ to C₄ alkyl groups;
R₂ is independently selected from the group consisting of C₂ to C₈ alkyl groups;
R₃ is independently selected from the group consisting of C₁ to C₁₂ alkyl groups;
R₄ is independently selected from the group consisting of —H and C₁ to C₁₂ alkyl groups;
R₅ is independently selected from the group consisting of C₂ to C₈ alkyl groups;
R₆ is independently selected from the group consisting of —H, and C₁ to C₁₂ alkyl groups; and
R₇ is independently selected from the group consisting of C₁ to C₁₂ alkyl groups.

8. The composition of claim 1, further comprising:
a helper lipid;
a structural lipid or sterol; and
a polymer-conjugated lipid, wherein the composition forms lipid nanoparticles.

9. The composition of claim 2, further comprising
a helper lipid;
a structural lipid or sterol; and
a polymer-conjugated lipid, wherein the composition forms lipid nanoparticles.

10. The composition of claim 8 wherein the helper lipid is selected from the group consisting of 1, 2-distearoyl-sn-glycero-3-phosphocholine (DSPC), 1,2-dioleoyl-sn-glycero-3-phosphoethanolamine (DOPE), 1,2-dilinoleoyl-sn-glycero-3-phosphocholine (DLPC), 1,2-dimyristoyl-sn-glycero-phosphocholine (DMPC), 1,2-dioleoyl-sn-glycero-3-phosphocholine (DOPC), 1,2-dipalmitoyl-sn-glycero-3-phosphocholine (DPPC), 1,2-diundecanoyl-sn-glycero-phosphocholine (DUPC), 1-palmitoyl-2-oleoyl-sn-glycero-3-phosphocholine (POPC), 1,2-di-O-octadecenyl-sn-glycero-3-phosphocholine (18:0 Diether PC), 1-oleoyl-2-cholesterylhemisuccinoyl-sn-glycero-3-phosphocholine (OChemsPC), 1-hexadecyl-sn-glycero-3-phosphocholine (C16 Lyso PC), 1,2-dilinolenoyl-sn-glycero-3-phosphocholine, 1,2-diarachidonoyl-sn-glycero-3-phosphocholine, 1,2-didocosahexaenoyl-sn-glycero-3-phosphocholine, 1,2-diphytanoyl-sn-glycero-3-phosphoethanolamine (ME 16.0 PE), 1,2-distearoyl-sn-glycero-3-phosphoethanolamine, 1,2-dilinoleoyl-sn-glycero-3-phosphoethanolamine, 1,2-dilinolenoyl-sn-glycero-3-phosphoethanolamine, 1,2-diarachidonoyl-sn-glycero-3-phosphoethanolamine, 1,2-didocosahexaenoyl-sn-glycero-3-phosphoethanolamine, 1, 2-dioleoyl-sn-glycero-3-phospho-rac-(1-glycerol) sodium salt (DOPG), sphingomyelin and combinations thereof.

11. The composition of claim 10, wherein the helper lipid is DSPC.

12. The composition of claim 10, wherein the helper lipid is DOPE.

13. The composition of claim 10, wherein the helper lipid is a combination of DSPC and DOPE.

14. The composition of claim 8, wherein the structural lipid or sterol is cholesterol or a derivative thereof.

15. The composition of claim 8, wherein the polymer-conjugated lipid is selected from the group consisting of 1-(monomethoxy-polyethyleneglycol)-2, 3-dimyristoylglycerol (PEG-DMG); pegylated phosphatidylethanoloamine (PEG-PE); 4-O-(2′,3′-di (tetradecanoyloxy) propyl-1-O-(ω-methoxy (polyethoxy) ethyl) butanedioate (PEG-S-DMG); ω-methoxy (polyethoxy) ethyl-N-(2,3-di (tetradecanoxy) propyl) carbamate; and 2,3-di(tetradecanoxy) propyl-N-(ω-methoxy (polyethoxy) ethyl) carbamate.

16. The composition of claim 8, wherein the ionizable lipid comprises from about 40-60 molar percent, the helper lipid comprises from about 10-20 molar percent, the sterol comprises from about 30-50 molar percent; and the conjugate lipid comprises from about 1-5 molar percent.

17. The composition of claim 8, wherein the lipid nanoparticle at least partially encapsulates a nucleic acid.

18. The composition of claim 17, wherein the nucleic acid is mRNA.

19. The composition of claim 8, further comprising a pharmaceutically acceptable excipient.

20. The composition of claim 19, wherein the composition is formulated for administration by injection or infusion.

21-23. (canceled)
